(19) United States
(12) Patent Application Publication
(10) Pub. No.: US 2022/0411802 A1
(43) Pub. Date: Dec. 29, 2022

(54) METHODS OF CANCER TREATMENT BY DELIVERY OF siRNAs AGAINST NSD3

(71) Applicant: SIRNAOMICS, INC., Gaithersburg, MD (US)

(72) Inventor: David M. EVANS, Gaithersburg, MD (US)

(21) Appl. No.: 17/697,717

(22) Filed: Mar. 17, 2022

Related U.S. Application Data
(60) Provisional application No. 63/162,497, filed on Mar. 17, 2021.

(51) Int. Cl. C12N 15/113; A61K 33/243; A61K 31/282; A61K 9/16

(52) U.S. Cl. CPC C12N 15/1137; A61K 33/243; A61K 31/282; A61K 9/1658; C12N 2310/14; C12N 2320/31; C12N 2320/32

(57) ABSTRACT

Compositions and methods are provided for the silencing of the NSD3 gene. Specifically, siRNA compositions are provided that contain siRNA molecules that target the wild-type NSD3 gene or the NSD3_{T1232A} mutant. Methods for using these compositions for treating cancer also are provided.

### CROSS REFERENCE TO RELATED PATENT APPLICATIONS

This application claims the benefit of and priority to U.S. Provisional Patent Application No. 63/162,497, filed Mar. 17, 2021, the contents of which are incorporated herein by reference in their entirety.

### BACKGROUND

Histone H3 lysine 36 (H3K36) methyltransferase (“NSD3”) is a regulator of lung squamous cell carcinoma (LUSC) tumorigenesis. Increased expression of NSD3 correlates with amplification of the gene. The gene sequence of NSD3 is shown in FIG. 1, and the start codon for translation is shown in bold and underline. An LUSC-associated variant NSD3(T1232A; Start base for mutation=3696; bases 3147-3765) shows increased catalytic activity for dimethylation of H3K36 (H3K36me2) in vitro and in vivo. The T1232A mutation affects auto-inhibition and increases accessibility of the histone H3 substrate. The location of the mutation is shown in FIG. 1 in bold underlined italics.

### SUMMARY OF THE INVENTION

Nanoparticle compositions are provided that contain an NSD3-silencing amount of an siRNA molecule that targets wild-type NSD3 or mutated NSD3. The siRNA that targets wild-type NSD3 contains a sequence selected from the group consisting of SEQ ID NOs:1-6 and the siRNA molecules shown in FIGS. 2 and 3. The siRNA that targets mutated NSD3 contains a sequence selected from the group consisting of SEQ ID NOs:7-12 (for 19mer siRNAs) and SEQ ID NO: 13-15 (for 25mer siRNAs). The composition may contain an HKP, for example HKP(+H).

Also provided are methods of treating a cancer in a subject, such as a human subject, suffering from the cancer, in which an effective amount of a nanoparticle composition as described above is administered to the subject. The cancer may be, for example, LUSC. In these methods the nanoparticle composition may be delivered systemically or intratumorally. In these methods an effective amount of a chemotherapy drug may be administered to the subject together with the nanoparticle composition. The administration of the chemotherapy drug may be substantially contemporaneous with the nanoparticle composition, or the chemotherapy drug may be administered prior to, or after the nanoparticle composition. The chemotherapy drug may be, for example, a platinum-containing drug, such as cisplatin, oxaloplatin, or carboplatin.

### BRIEF DESCRIPTION OF THE DRAWINGS

FIG. 1 shows the sequence of the NSD3 gene.

FIG. 2 shows the sequence of the sense strand of 25-mer siRNA molecules that target wild-type NSD3.

FIG. 3 shows the sequence of the sense strand of 19-mer siRNA molecules that target wild-type NSD3.

FIG. 4 (a) shows NSD3 siRNA #1-#14 screening in SK-MES1 cells, reverse transfection over 24 hours using SYBR Green qRT-PCR. Sequences 1 8, 10 and 12 demonstrate good silencing of the native NSD3 sequence.

FIG. 4 (b) shows a variety of siRNA sequences screened against NSD3 sequences.

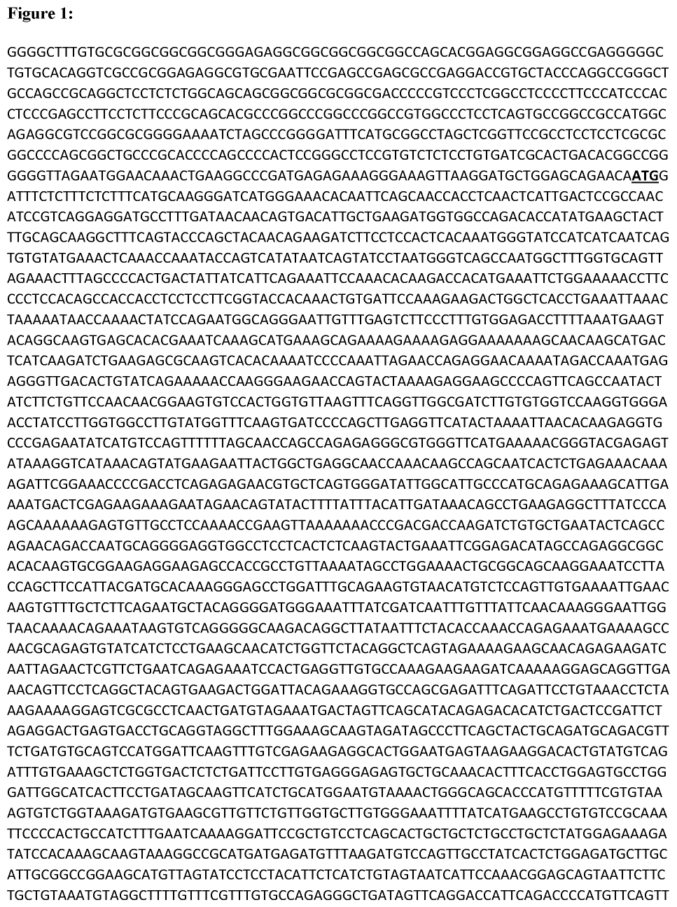

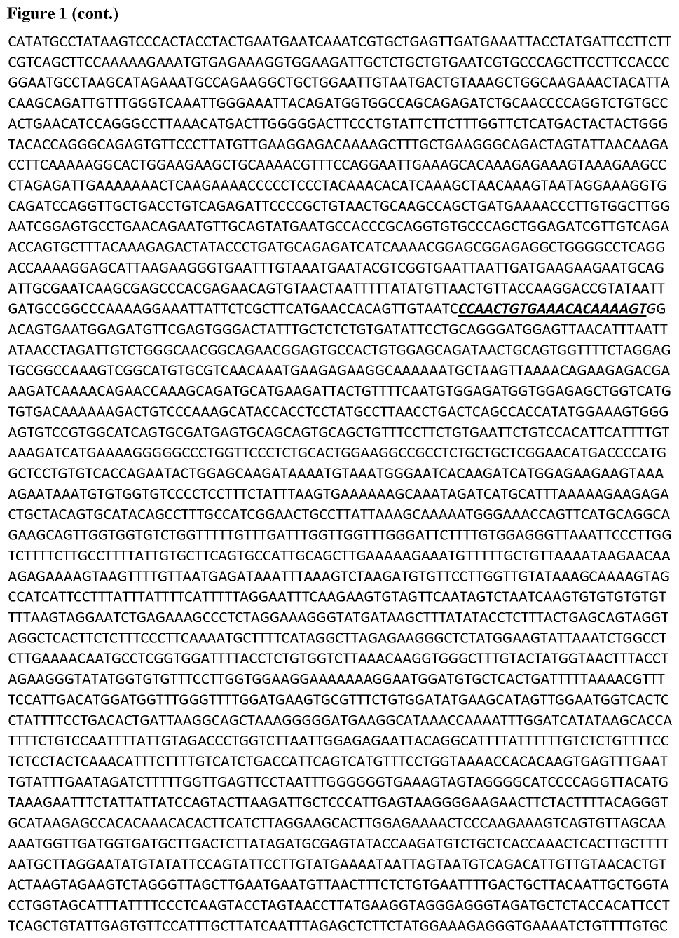

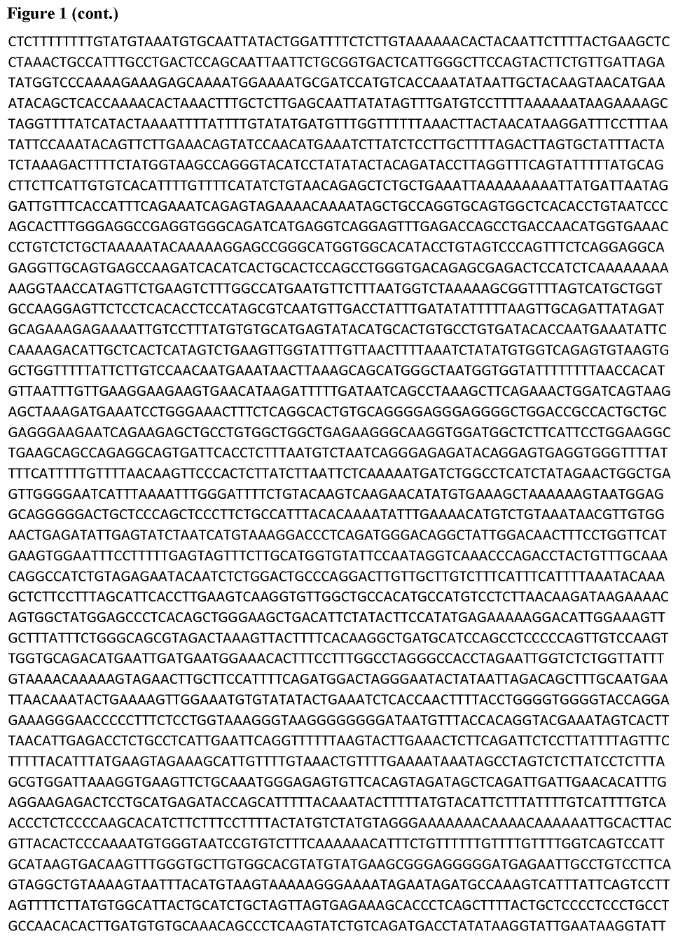

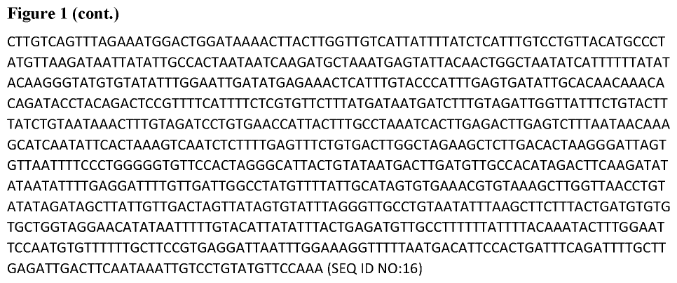

### DETAILED DESCRIPTION

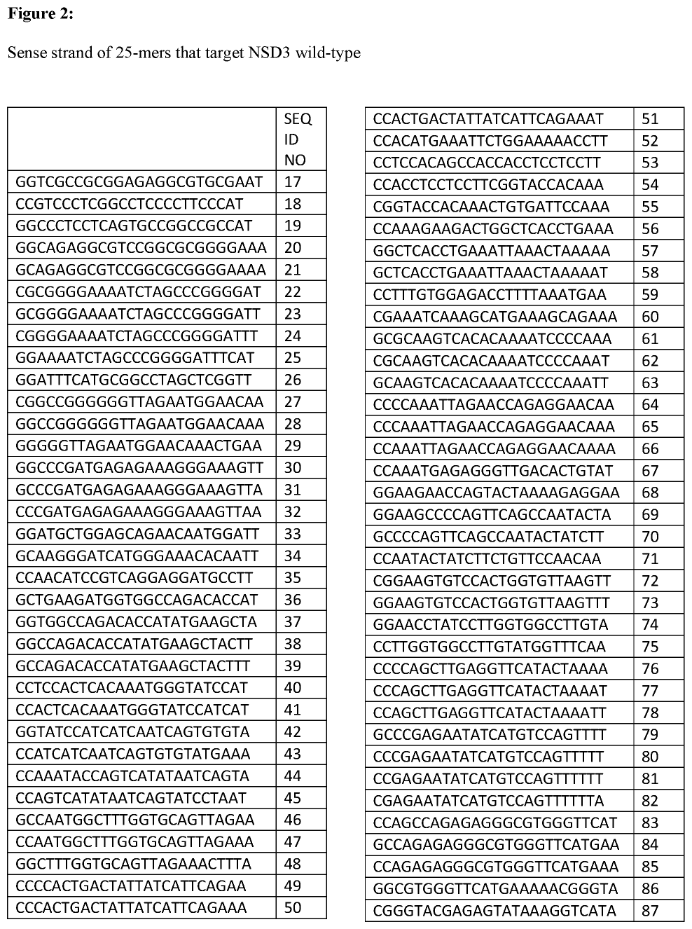

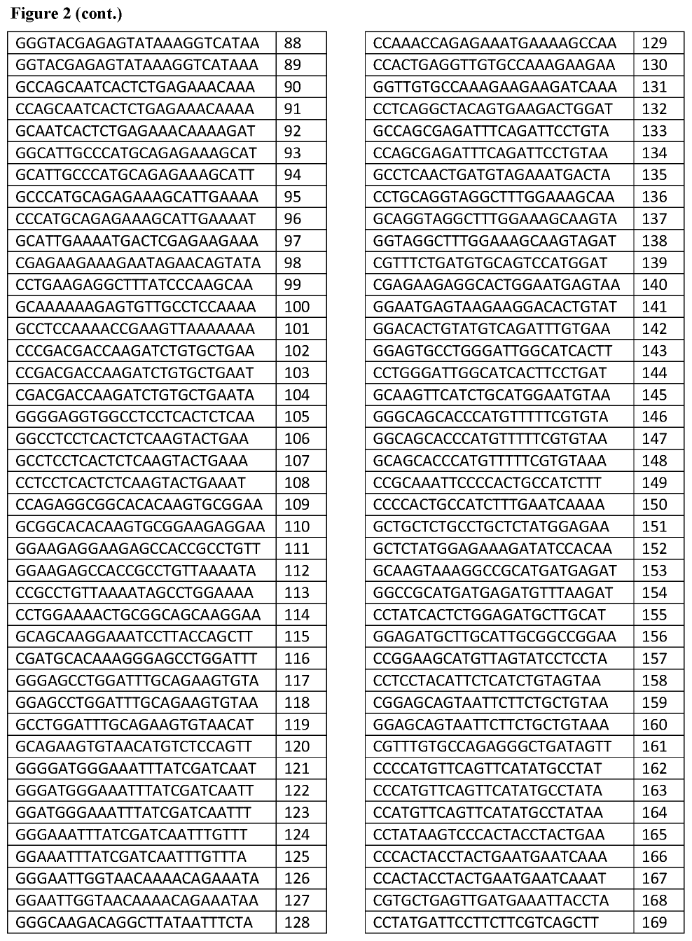

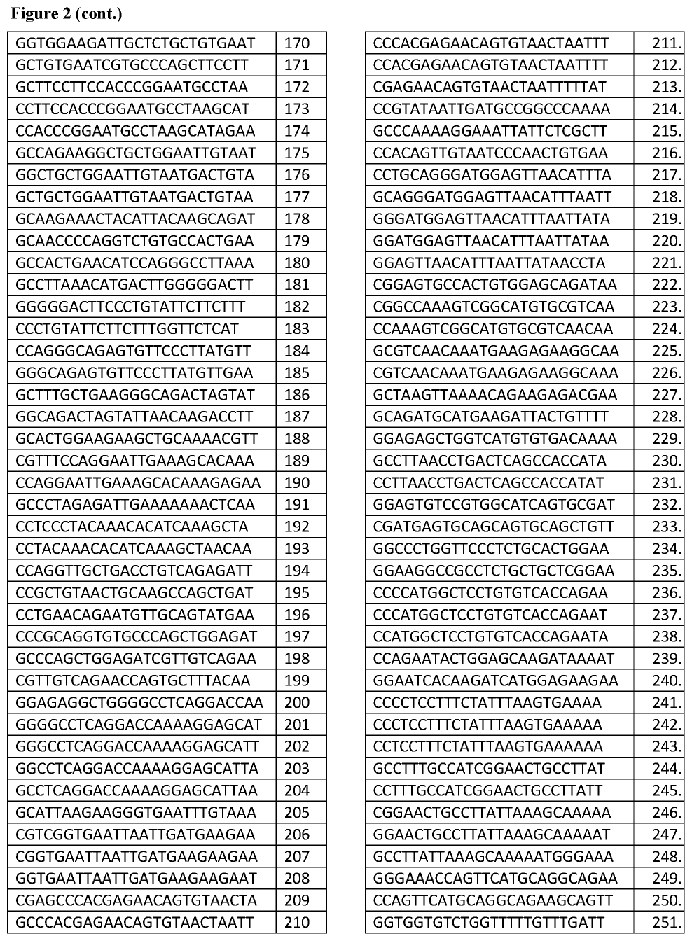

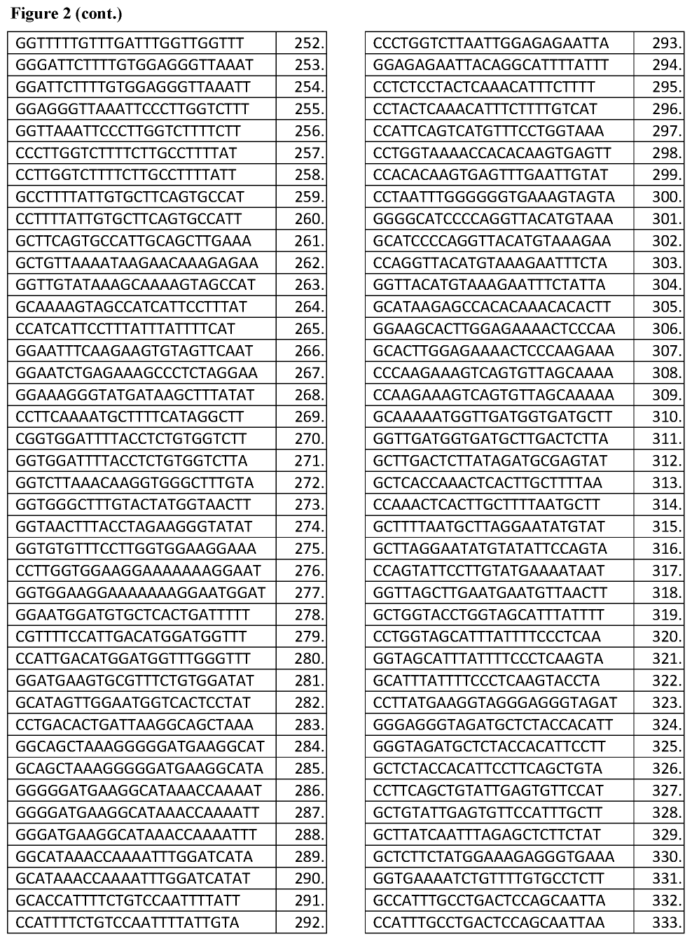

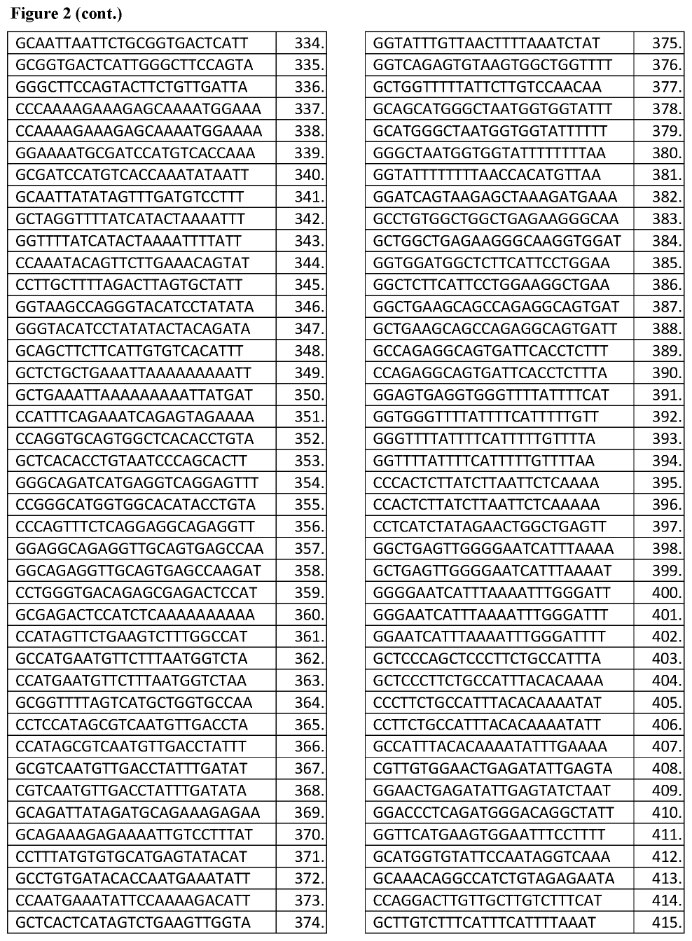

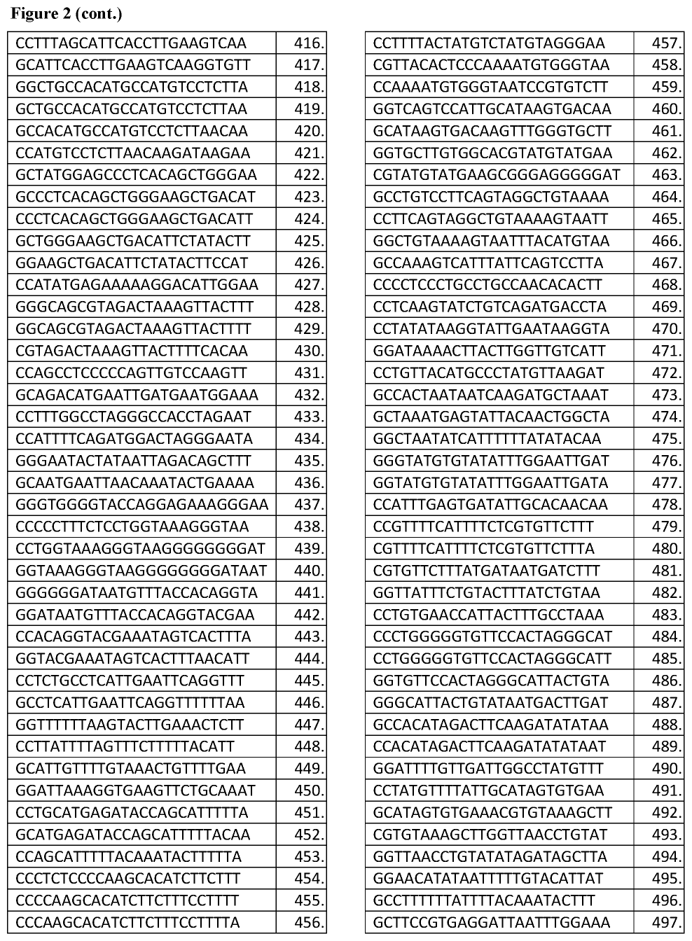

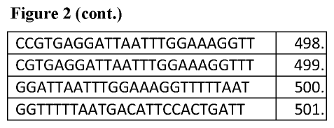

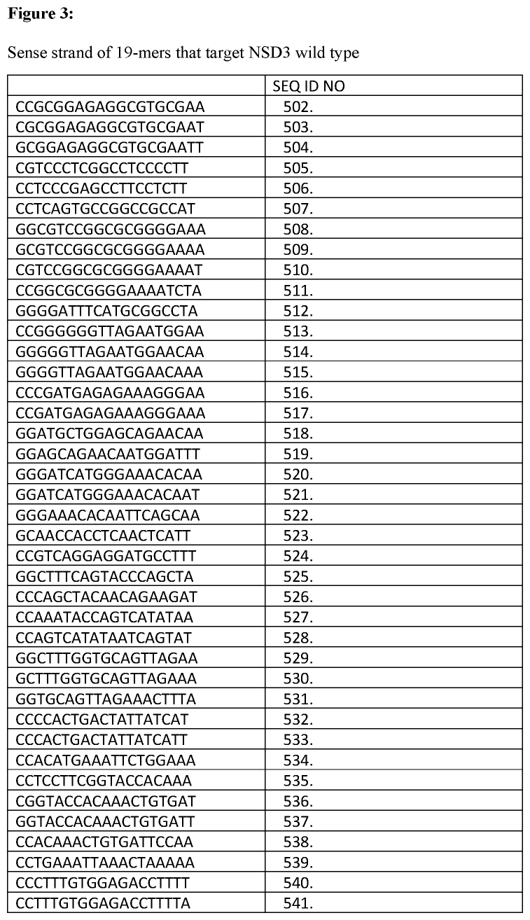

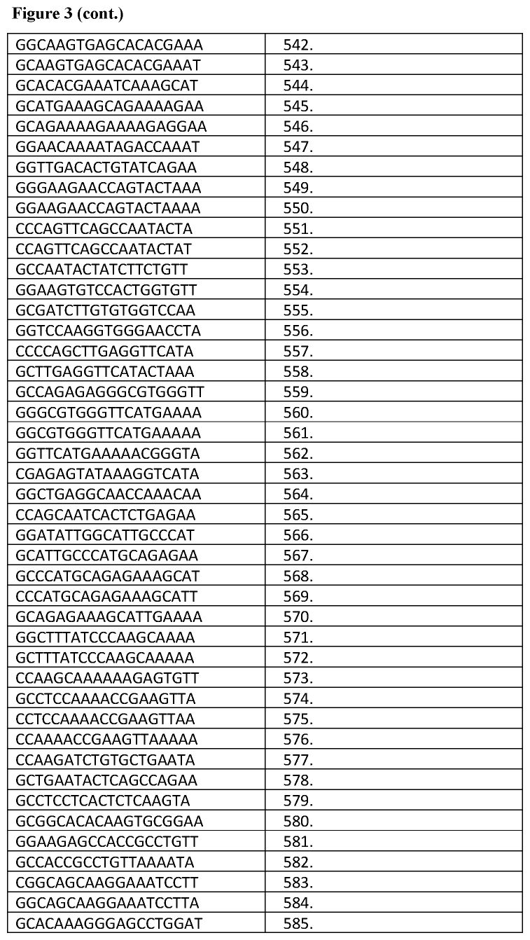

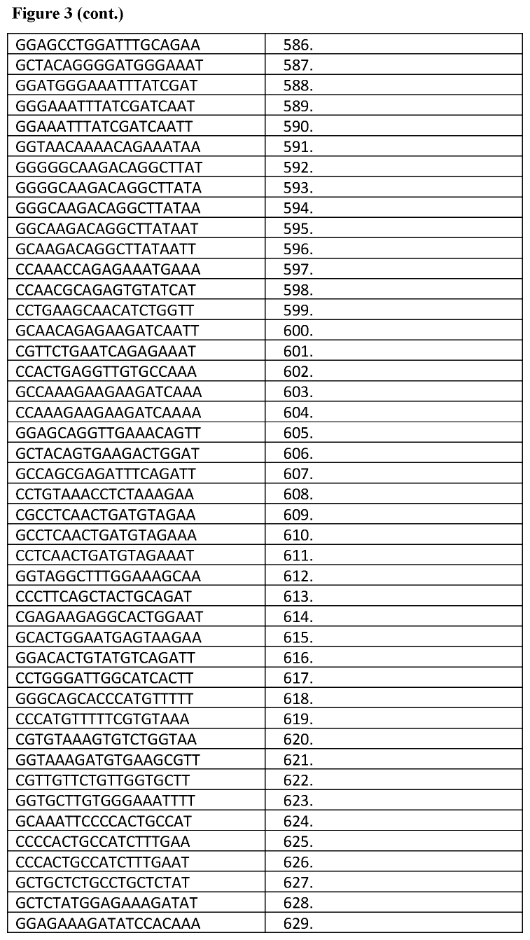

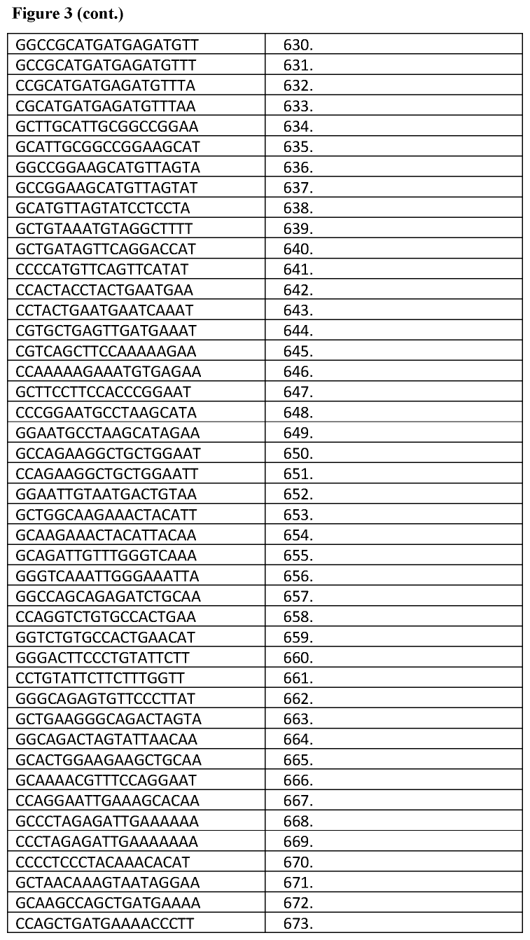

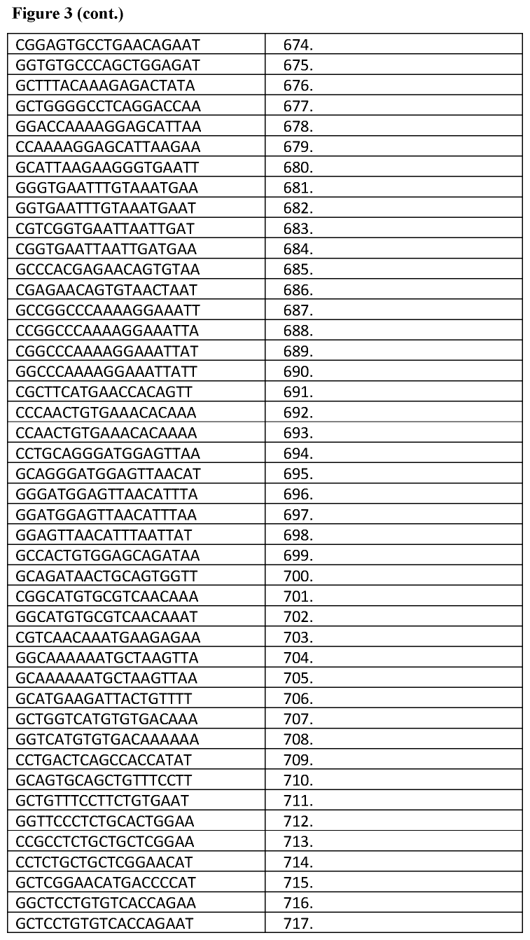

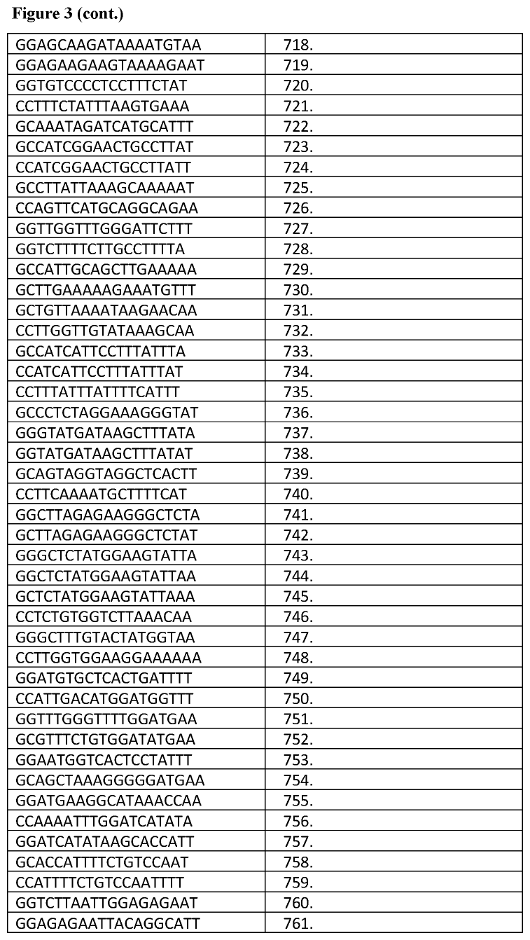

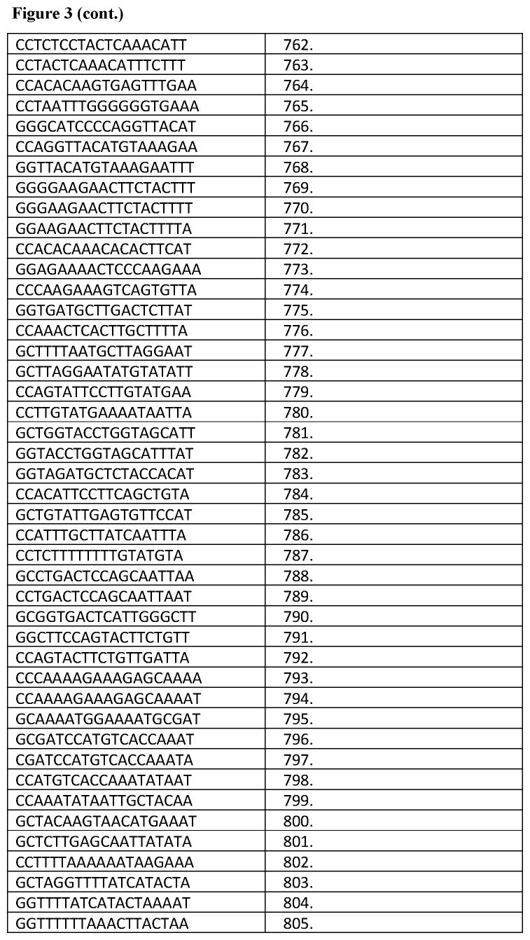

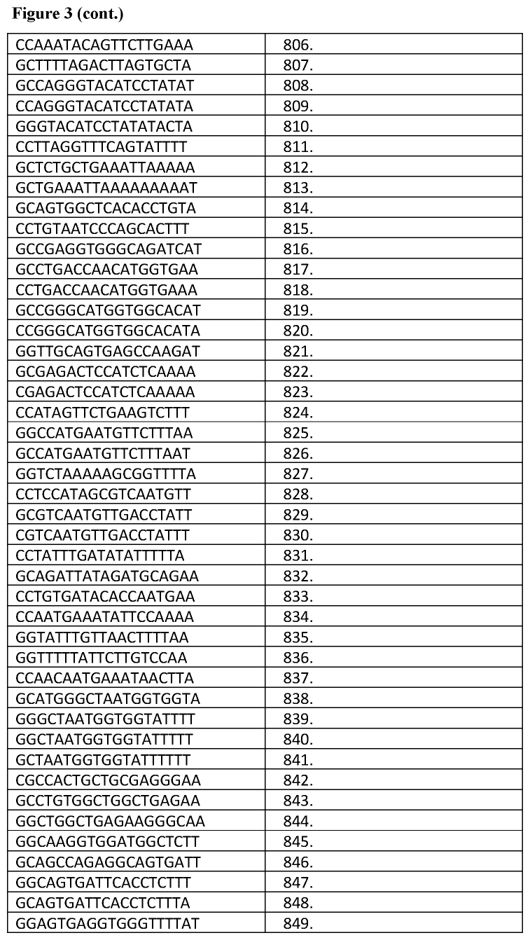

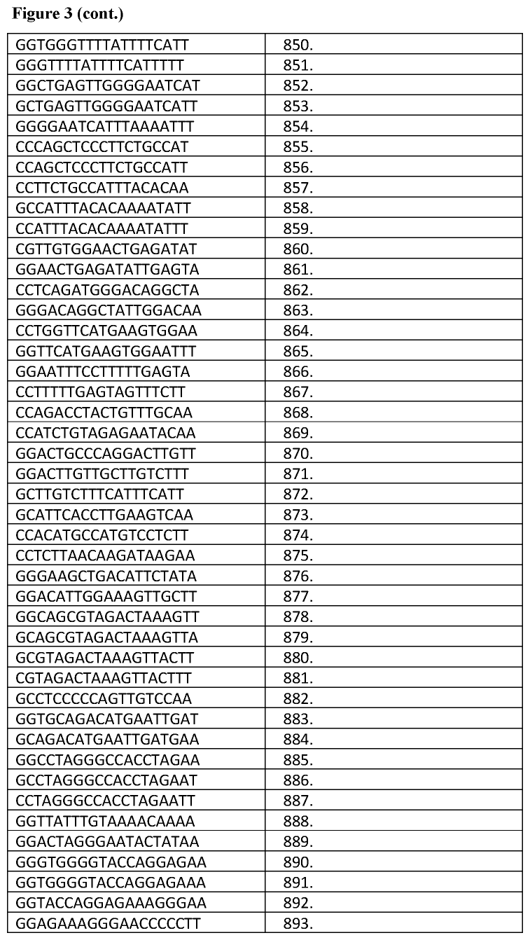

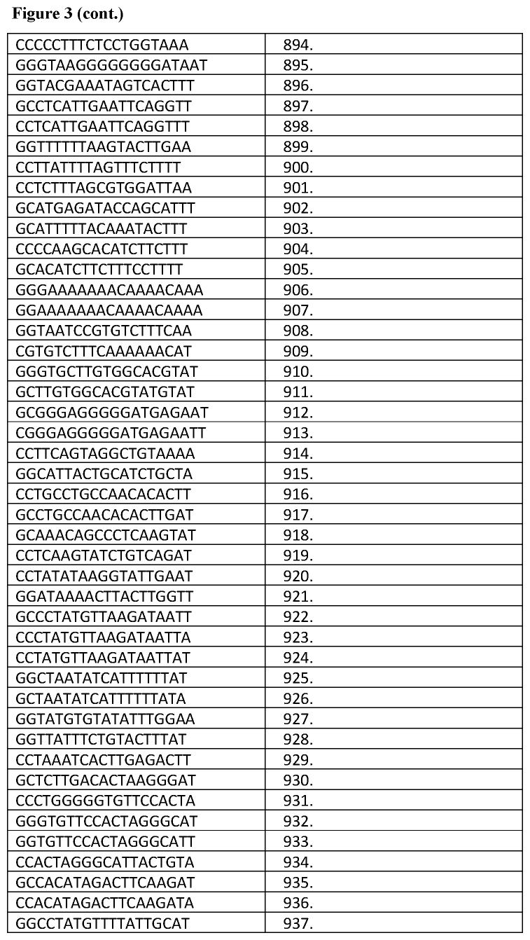

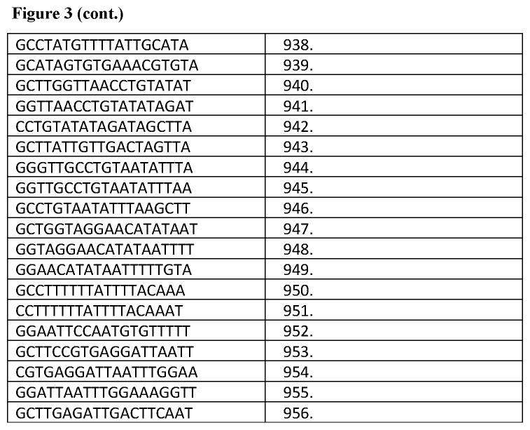

Compositions, including nanoparticle compositions, are provided that contain a NSD3-silencing amount of an siRNA molecule that targets NSD3. The siRNA that targets NSD3 may target either the wild-type sequence or the T1232A mutated protein. The sense strand of 25-mer duplexes that target wild-type NSD3 are shown in FIG. 2. The sense strand of duplexes that target wild-type NSD3 may contain the 19-mer sequences shown in FIG. 3. These siRNA molecules targeting NSD3 wild-type advantageously are 19-25 nucleotides in length and may have a one or two residue overhang at one or both ends or, advantageously, are blunt ended.

Specific examples of the sense strand of siRNA duplexes that target wild-type NSD3 include 19-25mer molecules that contain the sequences of SEQ ID NOs:1-6:

(SEQ ID NO: 1)
GGGATGGAGTTAACATTTA
(SEQ ID NO: 2)
GCTGTTTCCTTCTGTGAAT
(SEQ ID NO: 3)
CCTTGGTTGTATAAAGCAA
(SEQ ID NO: 4)
CCTTCAAAATGCTTTTCAT
(SEQ ID NO: 5)
CCTCTGTGGTCTTAAACAA
(SEQ ID NO: 6)
CCCACTGACTATTATCATTCAGAAA

The sense strand of 19-mer sequences that target the T1232A mutated NSD3 mRNA are shown below (SEQ ID NO:7-12). The sequence coding for the alanine 1232 is shown in bold.

(SEQ ID NO: 7)
CCAACTGTGAAGCACAAAA
(SEQ ID NO: 8)
CCAACTGTGAAGCCCAAAA
(SEQ ID NO: 9)
CCAACTGTGAAGCGCAAAA

The sense strand sequences shown below result in cleavage of the mRNA at the base corresponding between base 10-11 of the AS siRNA (base 9-10 of the SS for a 19mer siRNA and base 15-16 for a 25mer). This is of the codon recognizing the expression of the alanine of the T1232A mutation.

(SEQ ID NO: 10)
ACTGTGAAGCACAAAAGTG
(SEQ ID NO: 11)
ACTGTGAAGCCCAAAAGTG
(SEQ ID NO: 12)
ACTGTGAAGCGCAAAAGTG

The following 25mer siRNA sequences also result in cleavage of the mutated NSD3T1232A mRNA:

(SEQ ID NO: 13)
ATCCCAACTGTGAAGCACAAAAGTG
(SEQ ID NO: 14)
ATCCCAACTGTGAAGCCCAAAAGTG
(SEQ ID NO: 15)
ATCCCAACTGTGAAGCGCAAAAGTG

The siRNA molecules may be used as single duplex molecules, or two or more siRNA molecules that target NSD3 may be combined. Reference herein to the siRNA molecule of SEQ ID NO:X will be understood to refer to the duplex formed by the sense strand (SEQ ID NO:X) and the corresponding antisense strand.

The siRNA molecules may be formulated in nanoparticles for administration. The nanoparticles may contain one or more lipids, including neutral and cationic lipids. Advantageously, the nanoparticles contain an HKP (histidine-lysine polymer) as described, for example, in U.S. Pat. Nos. 7,163,695, 7,070,807, and 7,772,201, the contents of each of which are hereby incorporated in their entireties. Advantageously, the nanoparticles contain a highly-branched HKP as described in U.S. Pat. No. 7,772,201.

Also provided are methods of treating a cancer in a subject suffering from the cancer, in which an effective amount of a nanoparticle composition as described above is administered to the subject. The cancer may be, for example head and neck cancer, bladder cancer, pancreatic cancer, cholangiocarcinoma, lung cancer (NSCLC, SCLC, LUSC), colon cancer, glioblastoma, breast cancer, gastric adenocarcinomas, prostate cancer, ovarian carcinoma, cervical cancer, AML, ALL, myeloma or non-Hodgkins lymphoma. Advantageously the cancer is lung squamous cell carcinoma (LUSC). In these methods the composition may be delivered systemically or intratumorally.

Further provided are methods of treating cancer in a subject, in which the nanoparticle composition as described above is administered together with an effective amount of a chemotherapy drug. Examples of suitable chemotherapy drugs include platinum-containing drugs such as cisplatin, oxaloplatin, or carboplatin, docetaxel (Taxotere), gemcitabine (Gemzar), paclitaxel (Taxol), pemetrexed (Alimta), vinorelbine (Navelbine), Abraxane (Paclitaxel Albumin-stabilized Nanoparticle Formulation), Afatinib Dimaleate, Afinitor (Everolimus), Afinitor Disperz (Everolimus), Alecensa (Alectinib), Alectinib, Alimta (Pemetrexed Disodium), Alunbrig (Brigatinib), Atezolizumab, Avastin (Bevacizumab), Bevacizumab, Brigatinib, Capmatinib Hydrochloride, Carboplatin, Cemiplimab-rwlc, Ceritinib, Crizotinib, Cyramza (Ramucirumab), Dabrafenib Mesylate, Dacomitinib, Docetaxel, Doxorubicin Hydrochloride, Durvalumab, Entrectinib, Erlotinib Hydrochloride, Everolimus, Gavreto (Pralsetinib), Gefitinib, Gilotrif (Afatinib Dimaleate), Gemcitabine Hydrochloride, Gemzar (Gemcitabine Hydrochloride), Imfinzi (Durvalumab), Infugem (Gemcitabine Hydrochloride), Ipilimumab, Iressa (Gefitinib), Keytruda (Pembrolizumab), Libtayo (Cemiplimab-rwlc), Lorbrena (Lorlatinib), Lorlatinib, Mekinist (Trametinib Dimethyl Sulfoxide), Methotrexate Sodium, Mvasi (Bevacizumab), Necitumumab, Nivolumab, Opdivo (Nivolumab), Osimertinib Mesylate, Paclitaxel, Paclitaxel Albumin-stabilized Nanoparticle Formulation, Paraplat (Carboplatin), Paraplatin (Carboplatin), Pembrolizumab, Pemetrexed Disodium, Portrazza (Necitumumab), Pralsetinib, Ramucirumab, Retevmo (Selpercatinib), Rozlytrek (Entrectinib), Selpercatinib, Tabrecta (Capmatinib Hydrochloride), Tafinlar (Dabrafenib Mesylate), Tagrisso (Osimertinib Mesylate), Tarceva (Erlotinib Hydrochloride), Taxotere (Docetaxel), Tecentriq (Atezolizumab), Tepmetko (Tepotinib Hydrochloride), Tepotinib Hydrochloride, Trametinib Dimethyl Sulfoxide, Trexall (Methotrexate Sodium), Vizimpro (Dacomitinib), Vinorelbine Tartrate, Xalkori (Crizotinib), Yervoy (Ipilimumab), Zirabev (Bevacizumab), Zykadia (Ceritinib), Drug Combinations Used to Treat Non-Small Cell Lung Cancer, CARBOPLATIN-TAXOL, GEMCITABINE-CISPLATIN, Drugs Approved for Small Cell Lung Cancer, Afinitor (Everolimus), Atezolizumab, Doxorubicin Hydrochloride, Durvalumab, Etopophos (Etoposide Phosphate), Etoposide, Etoposide Phosphate, Everolimus, Hycamtin (Topotecan Hydrochloride), Imfinzi (Durvalumab), Lurbinectedin, Methotrexate Sodium, Nivolumab, Opdivo (Nivolumab), Tecentriq (Atezolizumab), Topotecan Hydrochloride, and Trexall (Methotrexate Sodium).

As used herein, silencing a gene means reducing the concentration of the mRNA transcript of that gene such that the concentration of the protein product of that gene is reduced by at least 10%, at least 20%, at least 30%, at least 40%, at least 50%, at least 70%, at least 80% or at least 90% or more.

Formation of Nanoparticles Containing siRNAs Targeting NSD3.

The siRNA molecules containing the molecules described above advantageously are formulated into nanoparticles for administration to a subject. Various methods of nanoparticle formation are well known in the art. See, for example, Babu et al., IEEE Trans Nanobioscience, 15: 849-863 (2016).

Advantageously, the nanoparticles are formed using one or more histidine/lysine (HKP) copolymers. Suitable HKP copolymers are described in WO/2001/047496, WO/2003/090719, and WO/2006/060182, the contents of each of which are incorporated herein in their entireties. HKP copolymers form a nanoparticle containing an siRNA molecule, typically 100-400 nm in diameter. HKP and HKP(+H) both have a lysine backbone (three lysine residues) where the lysine side chain ε-amino groups and the N-terminus are coupled to [KH₃]₄K (for HKP) or KH₃KH₄[KH₃]₂K (for HKP(+H). The branched HKP carriers can be synthesized by methods that are well-known in the art including, for example, solid-phase peptide synthesis.

Methods of forming nanoparticles are well known in the art. Babu et al., supra. Advantageously, nanoparticles may be formed using a microfluidic mixer system, in which an siRNA targeting NSD3 is mixed with one or more HKP polymers at a fixed flow rate. The flow rate can be varied to vary the size of the nanoparticles produced.

Determination of Efficacy of the siRNA Molecules

Depending on the particular target NSD3 RNA sequences and the dose of the nanoparticle composition delivers, partial or complete loss of function for the NSD3 RNAs may be observed. A reduction or loss of RNA levels or expression (either NSD3 RNA expression or encoded polypeptide expression) in at least 50%, 60%, 70%, 80%, 90%, 95% or 99% or more of targeted cells is exemplary. Inhibition of NSD3 RNA levels or expression refers to the absence (or observable decrease) in the level of NSD3 RNA or NSD3 RNA-encoded protein. Specificity refers to the ability to inhibit the NSD3 RNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target NSD3 RNA sequence(s) by the dsRNA agents of the invention also can be measured based upon the effect of administration of such dsRNA agents upon development/progression of a NSD3 associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, or 10⁷-fold reduction in cancer cell levels could be achieved via administration of the nanoparticle composition to cells, a tissue, or a subject. The subject may be a mammal, such as a human.

Pharmaceutical Compositions and Methods of Administration

The nanoparticle compositions may be further formulated as a pharmaceutical composition using methods that are well known in the art. The composition may be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, trehalose, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Determination of Dosage and Toxicity

Toxicity and therapeutic efficacy of the compositions may be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds advantageously exhibit high therapeutic indices

Data from cell culture assays and animal studies can be used in formulating a range of dosage for use in humans. The dosage of the compositions advantageously is within a range of circulating concentrations that include the ED₅₀ with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC₅₀ (i.e., the concentration of the composition which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a composition as described herein can be in the range of approximately 1 pg to 1000 mg. For example, 10, 30, 100, or 1000 pg, or 10, 30, 100, or 1000 ng, or 10, 30, 100, or 1000 μg, or 10, 30, 100, or 1000 mg, or 1-5 g of the compositions can be administered. In general, a suitable dosage unit of the compositions described herein will be in the range of 0.001 to 0.25 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical composition can be administered once daily, or may be dosed in dosage units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

The compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition as described herein may include a single treatment or, advantageously, can include a series of treatments.

As used herein, a pharmacologically or therapeutically effective amount refers to that amount of an siRNA composition effective to produce the intended pharmacological, therapeutic or preventive result. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30% reduction in a measurable parameter associated with a disease or disorder, a therapeutically effective amount of a drug for the treatment of that disease or disorder is the amount necessary to effect at least a 30% reduction in that parameter.

Suitably formulated pharmaceutical compositions as described herein may be administered by means known in the art such as by parenteral routes, including intravenous, intramuscular, intraperitoneal, subcutaneous, transdermal, airway (aerosol), rectal, vaginal and topical (including buccal and sublingual) administration. Advantageously, the pharmaceutical compositions are administered by intravenous or intraparenteral infusion or injection.

Methods of Treatment

The compositions described herein may be used to treat proliferative diseases, such as cancer, characterized by expression, and particularly altered expression, of NSD3. Exemplary cancers include liver cancer (e.g. hepatocellular carcinoma or HCC), lung cancer (e.g., LUSC), colorectal cancer, prostate cancer, pancreatic cancer, ovarian cancer, cervical cancer, brain cancer (e.g., glioblastoma), renal cancer (e.g., papillary renal carcinoma), stomach cancer, esophageal cancer, medulloblastoma, thyroid carcinoma, rhabdomyosarcoma, osteosarcoma, squamous cell carcinoma (e.g., oral squamous cell carcinoma), melanoma, breast cancer, and hematopoietic disorders (e.g., leukemias and lymphomas, and other immune cell-related disorders). Other cancers include bladder, cervical (uterine), endometrial (uterine), head and neck, and oropharyngeal cancers. Advantageously, the cancer is head and neck cancer, bladder cancer, pancreatic cancer, cholangiocarcinoma, lung cancer (LUSC, NSCLC and SCLC), colon cancer, glioblastoma, breast cancer, gastric adenocarcinomas, prostate cancer, ovarian carcinoma, cervical cancer, AML, ALL, myeloma or non-Hodgkins lymphoma.

The compositions may be administered as described above and, advantageously may be delivered systemically or intratumorally. The compositions may be administered as a monotherapy, i.e. in the absence of another treatment, or may be administered as part of a combination regimen that includes one or more additional medications. Advantageously, when used as part of a combination regimen that includes an effective amount of at least one additional chemotherapy drug, as described above.

### Example 1: Screening of NSD3 siRNA Sequences in SK-MES1 Cells

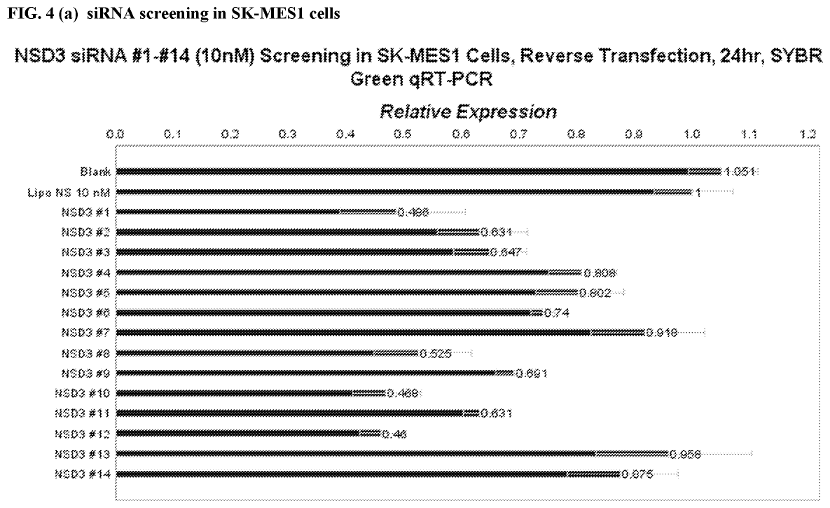

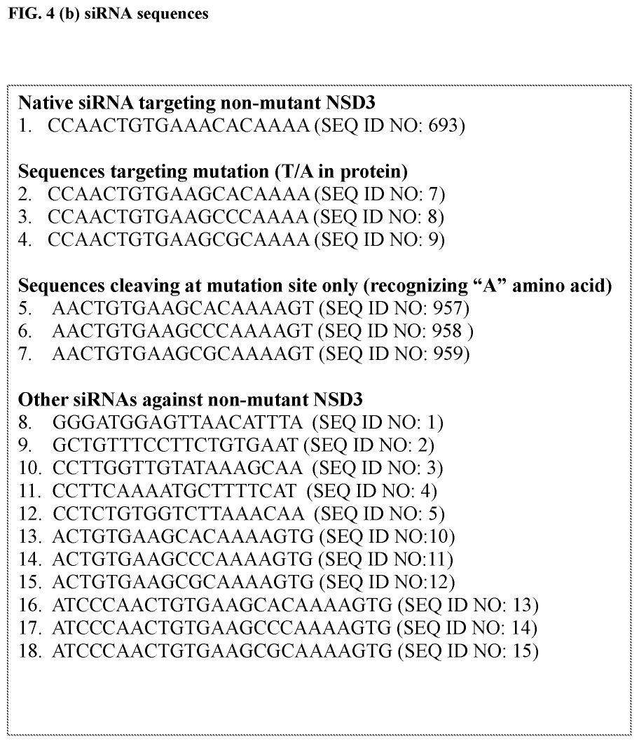

FIG. 4 (a) shows the screening of 14 NSD3 siRNA sequences (#1-#14, FIG. 4(b)) in SK-MES1 cells using SYBR Green qRT-PCR with reverse transfection over 24 hours. Out of the siRNA sequences that were designed against the mutant form of the NSD3 (#5-7), nos. 5 and 7 show the lowest degree of silencing of the native NSD3 sequence. These sequences may only recognize the mutant gene sequence. Sequence nos. 2 and 3 against the T/A mutation also show activity against the native NSD3 sequence. Sequence nos. 1, 8, 10 and 12 (aimed at the non-mutant gene sequence) show very good silencing of the native NSD3.

What is claimed is:

1. A nanoparticle composition comprising a NSD3-silencing amount of an siRNA molecule that targets wild-type NSD3 or mutated NSD3 wherein said siRNA that targets wild-type NSD3 comprises a sequence selected from the group consisting of SEQ ID NOs:1-6 and the siRNA molecules shown in FIGS. 2 and 3, and the siRNA that targets mutated NSD3 comprises a sequence selected from the group consisting of SEQ ID NOs:7-12 (for 19mer siRNAs) and 13-15 (for 25mer siRNAs).

2. The composition according to claim 1, comprising an siRNA that targets wild-type NSD3.

3. The composition according to claim 2 wherein said siRNA that targets wild-type NSD3 is selected from the group consisting of SEQ ID NOs:1-6.

4. The composition according to claim 2 comprising an siRNA that targets wild-type NSD3 comprising a sequence selected from the group consisting of the siRNA sequences shown in FIGS. 2 and 3.

5. The composition according to claim 1 comprising an siRNA that targets mutated NSD3, wherein said siRNA comprises a sequence selected from the group consisting of SEQ ID NOs:7-12 or 13-15.

6. The composition according to any preceding claim wherein the nanoparticle comprises an HKP.

7. The composition according to any preceding claim wherein the HKP is HKP(+H).

8. A method of treating a cancer in a subject suffering from said cancer, comprising administering to said subject an effective amount of a composition according to any preceding claim.

9. The method according to claim 8, wherein said cancer is LUSC.

10. The method according to claim 8, wherein said composition is delivered systemically or intratumorally.

11. The method according to any of claim 8, further comprising administering an effective amount of a chemotherapy drug.

12. The method according to claim 11 wherein said chemotherapy drug is a platinum-containing drug.

13. The method according to claim 12 wherein said platinum-containing drug is cisplatin, oxaloplatin, or carboplatin.


SEQUENCE LISTING (956 sequences)

SEQ ID NO: 1 - DNA, 19 bp, Artificial Sequence
  combination DNA-RNA, siRNA targeting wild-type NSD3
gggatggagttaacattta

SEQ ID NO: 2 - DNA, 19 bp, Artificial Sequence
  combination DNA-RNA, siRNA targeting wild-type NSD3
gctgtttccttctgtgaat

SEQ ID NO: 3 - DNA, 19 bp, Artificial Sequence
  combination DNA-RNA, siRNA targeting wild-type NSD3
ccttggttgtataaagcaa

SEQ ID NO: 4 - DNA, 19 bp, Artificial Sequence
  combination DNA-RNA, siRNA targeting wild-type NSD3
ccttcaaaatgcttttcat

SEQ ID NO: 5 - DNA, 19 bp, Artificial Sequence
  combination DNA-RNA, siRNA targeting wild-type NSD3
cctctgtggtcttaaacaa

SEQ ID NO: 6 - DNA, 25 bp, Artificial Sequence
  combination DNA-RNA, siRNA targeting wild-type NSD3
cccactgactattatcattcagaaa

SEQ ID NO: 7 - DNA, 19 bp, Artificial Sequence
  combination DNA-RNA, 19-mer sequences that target the T1232A mutated NSD3 mRNA
ccaactgtgaagcacaaaa

SEQ ID NO: 8 - DNA, 19 bp, Artificial Sequence
  combination DNA-RNA, 19-mer sequences that target the T1232A mutated NSD3 mRNA
ccaactgtgaagcccaaaa

SEQ ID NO: 9 - DNA, 19 bp, Artificial Sequence
  combination DNA-RNA, 19-mer sequences that target the T1232A mutated NSD3 mRNA
ccaactgtgaagcgcaaaa

SEQ ID NO: 10 - DNA, 19 bp, Artificial Sequence
  combination DNA-RNA, 19-mer sequences that target the T1232A mutated NSD3 mRNA
actgtgaagcacaaaagtg

SEQ ID NO: 11 - DNA, 19 bp, Artificial Sequence
  combination DNA-RNA, 19-mer sequences that target the T1232A mutated NSD3 mRNA
actgtgaagcccaaaagtg

SEQ ID NO: 12 - DNA, 19 bp, Artificial Sequence
  combination DNA-RNA, 19-mer sequences that target the T1232A mutated NSD3 mRNA
actgtgaagcgcaaaagtg

SEQ ID NO: 13 - DNA, 25 bp, Artificial Sequence
  combination DNA-RNA, 25mer siRNA sequences also result in cleavage of the mutated NSD3T1232A mRNA
atcccaactgtgaagcacaaaagtg

SEQ ID NO: 14 - DNA, 25 bp, Artificial Sequence
  combination DNA-RNA, 25mer siRNA sequences also result in cleavage of the mutated NSD3T1232A mRNA
atcccaactgtgaagcccaaaagtg

SEQ ID NO: 15 - DNA, 25 bp, Artificial Sequence
  combination DNA-RNA, 25mer siRNA sequences also result in cleavage of the mutated NSD3T1232A mRNA
atcccaactgtgaagcgcaaaagtg

SEQ ID NO: 16 - DNA, 10768 bp, homo sapiens
ggggctttgtgcgcggcggcggcgggagaggcggcggcggcggccagcacggaggcggaggccgagggggctgtgcacaggtcgccgcggagaggcgtgcgaattccgagccgagcgccgaggaccgtgctacccaggccgggctgccagccgcaggctcctctctggcagcagcggcggcgcggcgacccccgtccctcggcctccccttcccatcccacctcccgagccttcctcttcccgcagcacgcccggcccggcccggccgtggccctcctcagtgccggccgccatggcagaggcgtccggcgcggggaaaatctagcccggggatttcatgcggcctagctcggttccgcctcctcctcgcgcggccccagcggctgcccgcaccccagccccactccgggcctccgtgtctctcctgtgatcgcactgacacggccggggggttagaatggaacaaactgaaggcccgatgagagaaagggaaagttaaggatgctggagcagaacaatggatttctctttctctttcatgcaagggatcatgggaaacacaattcagcaaccacctcaactcattgactccgccaacatccgtcaggaggatgcctttgataacaacagtgacattgctgaagatggtggccagacaccatatgaagctactttgcagcaaggctttcagtacccagctacaacagaagatcttcctccactcacaaatgggtatccatcatcaatcagtgtgtatgaaactcaaaccaaataccagtcatataatcagtatcctaatgggtcagccaatggctttggtgcagttagaaactttagccccactgactattatcattcagaaattccaaacacaagaccacatgaaattctggaaaaaccttcccctccacagccaccacctcctccttcggtaccacaaactgtgattccaaagaagactggctcacctgaaattaaactaaaaataaccaaaactatccagaatggcagggaattgtttgagtcttccctttgtggagaccttttaaatgaagtacaggcaagtgagcacacgaaatcaaagcatgaaagcagaaaagaaaagaggaaaaaaagcaacaagcatgactcatcaagatctgaagagcgcaagtcacacaaaatccccaaattagaaccagaggaacaaaatagaccaaatgagagggttgacactgtatcagaaaaaccaagggaagaaccagtactaaaagaggaagccccagttcagccaatactatcttctgttccaacaacggaagtgtccactggtgttaagtttcaggttggcgatcttgtgtggtccaaggtgggaacctatccttggtggccttgtatggtttcaagtgatccccagcttgaggttcatactaaaattaacacaagaggtgcccgagaatatcatgtccagttttttagcaaccagccagagagggcgtgggttcatgaaaaacgggtacgagagtataaaggtcataaacagtatgaagaattactggctgaggcaaccaaacaagccagcaatcactctgagaaacaaaagattcggaaaccccgacctcagagagaacgtgctcagtgggatattggcattgcccatgcagagaaagcattgaaaatgactcgagaagaaagaatagaacagtatacttttatttacattgataaacagcctgaagaggctttatcccaagcaaaaaagagtgttgcctccaaaaccgaagttaaaaaaacccgacgaccaagatctgtgctgaatactcagccagaacagaccaatgcaggggaggtggcctcctcactctcaagtactgaaattcggagacatagccagaggcggcacacaagtgcggaagaggaagagccaccgcctgttaaaatagcctggaaaactgcggcagcaaggaaatccttaccagcttccattacgatgcacaaagggagcctggatttgcagaagtgtaacatgtctccagttgtgaaaattgaacaagtgtttgctcttcagaatgctacaggggatgggaaatttatcgatcaatttgtttattcaacaaagggaattggtaacaaaacagaaataagtgtcagggggcaagacaggcttataatttctacaccaaaccagagaaatgaaaagccaacgcagagtgtatcatctcctgaagcaacatctggttctacaggctcagtagaaaagaagcaacagagaagatcaattagaactcgttctgaatcagagaaatccactgaggttgtgccaaagaagaagatcaaaaaggagcaggttgaaacagttcctcaggctacagtgaagactggattacagaaaggtgccagcgagatttcagattcctgtaaacctctaaagaaaaggagtcgcgcctcaactgatgtagaaatgactagttcagcatacagagacacatctgactccgattctagaggactgagtgacctgcaggtaggctttggaaagcaagtagatagcccttcagctactgcagatgcagacgtttctgatgtgcagtccatggattcaagtttgtcgagaagaggcactggaatgagtaagaaggacactgtatgtcagatttgtgaaagctctggtgactctctgattccttgtgagggagagtgctgcaaacactttcacctggagtgcctgggattggcatcacttcctgatagcaagttcatctgcatggaatgtaaaactgggcagcacccatgtttttcgtgtaaagtgtctggtaaagatgtgaagcgttgttctgttggtgcttgtgggaaattttatcatgaagcctgtgtccgcaaattccccactgccatctttgaatcaaaaggattccgctgtcctcagcactgctgctctgcctgctctatggagaaagatatccacaaagcaagtaaaggccgcatgatgagatgtttaagatgtccagttgcctatcactctggagatgcttgcattgcggccggaagcatgttagtatcctcctacattctcatctgtagtaatcattccaaacggagcagtaattcttctgctgtaaatgtaggcttttgtttcgtttgtgccagagggctgatagttcaggaccattcagaccccatgttcagttcatatgcctataagtcccactacctactgaatgaatcaaatcgtgctgagttgatgaaattacctatgattccttcttcgtcagcttccaaaaagaaatgtgagaaaggtggaagattgctctgctgtgaatcgtgcccagcttccttccacccggaatgcctaagcatagaaatgccagaaggctgctggaattgtaatgactgtaaagctggcaagaaactacattacaagcagattgtttgggtcaaattgggaaattacagatggtggccagcagagatctgcaaccccaggtctgtgccactgaacatccagggccttaaacatgacttgggggacttccctgtattcttctttggttctcatgactactactgggtacaccagggcagagtgttcccttatgttgaaggagacaaaagctttgctgaagggcagactagtattaacaagaccttcaaaaaggcactggaagaagctgcaaaacgtttccaggaattgaaagcacaaagagaaagtaaagaagccctagagattgaaaaaaactcaagaaaaccccctccctacaaacacatcaaagctaacaaagtaataggaaaggtgcagatccaggttgctgacctgtcagagattccccgctgtaactgcaagccagctgatgaaaacccttgtggcttggaatcggagtgcctgaacagaatgttgcagtatgaatgccacccgcaggtgtgcccagctggagatcgttgtcagaaccagtgctttacaaagagactataccctgatgcagagatcatcaaaacggagcggagaggctggggcctcaggaccaaaaggagcattaagaagggtgaatttgtaaatgaatacgtcggtgaattaattgatgaagaagaatgcagattgcgaatcaagcgagcccacgagaacagtgtaactaatttttatatgttaactgttaccaaggaccgtataattgatgccggcccaaaaggaaattattctcgcttcatgaaccacagttgtaatcccaactgtgaaacacaaaagtggacagtgaatggagatgttcgagtgggactatttgctctctgtgatattcctgcagggatggagttaacatttaattataacctagattgtctgggcaacggcagaacggagtgccactgtggagcagataactgcagtggttttctaggagtgcggccaaagtcggcatgtgcgtcaacaaatgaagagaaggcaaaaaatgctaagttaaaacagaagagacgaaagatcaaaacagaaccaaagcagatgcatgaagattactgttttcaatgtggagatggtggagagctggtcatgtgtgacaaaaaagactgtcccaaagcataccacctcctatgccttaacctgactcagccaccatatggaaagtgggagtgtccgtggcatcagtgcgatgagtgcagcagtgcagctgtttccttctgtgaattctgtccacattcattttgtaaagatcatgaaaagggggccctggttccctctgcactggaaggccgcctctgctgctcggaacatgaccccatggctcctgtgtcaccagaatactggagcaagataaaatgtaaatgggaatcacaagatcatggagaagaagtaaaagaataaatgtgtggtgtcccctcctttctatttaagtgaaaaaagcaaatagatcatgcatttaaaaagaagagactgctacagtgcatacagcctttgccatcggaactgccttattaaagcaaaaatgggaaaccagttcatgcaggcagaagcagttggtggtgtctggtttttgtttgatttggttggtttgggattcttttgtggagggttaaattcccttggtcttttcttgccttttattgtgcttcagtgccattgcagcttgaaaaagaaatgtttttgctgttaaaataagaacaaagagaaaagtaagttttgttaatgagataaatttaaagtctaagatgtgttccttggttgtataaagcaaaagtagccatcattcctttatttattttcatttttaggaatttcaagaagtgtagttcaatagtctaatcaagtgtgtgtgtgttttaagtaggaatctgagaaagccctctaggaaagggtatgataagctttatatacctctttactgagcagtaggtaggctcacttctctttcccttcaaaatgcttttcataggcttagagaagggctctatggaagtattaaatctggcctcttgaaaacaatgcctcggtggattttacctctgtggtcttaaacaaggtgggctttgtactatggtaactttacctagaagggtatatggtgtgtttccttggtggaaggaaaaaaaggaatggatgtgctcactgatttttaaaacgttttccattgacatggatggtttgggttttggatgaagtgcgtttctgtggatatgaagcatagttggaatggtcactcctattttcctgacactgattaaggcagctaaagggggatgaaggcataaaccaaaatttggatcatataagcaccattttctgtccaattttattgtagaccctggtcttaattggagagaattacaggcattttattttttgtctctgttttcctctcctactcaaacatttcttttgtcatctgaccattcagtcatgtttcctggtaaaaccacacaagtgagtttgaattgtatttgaatagatctttttggttgagttcctaatttggggggtgaaagtagtaggggcatccccaggttacatgtaaagaatttctattattatccagtacttaagattgctcccattgagtaaggggaagaacttctacttttacagggtgcataagagccacacaaacacacttcatcttaggaagcacttggagaaaactcccaagaaagtcagtgttagcaaaaatggttgatggtgatgcttgactcttatagatgcgagtataccaagatgtctgctcaccaaactcacttgcttttaatgcttaggaatatgtatattccagtattccttgtatgaaaataattagtaatgtcagacattgttgtaacactgtactaagtagaagtctagggttagcttgaatgaatgttaactttctctgtgaattttgactgcttacaattgctggtacctggtagcatttattttccctcaagtacctagtaaccttatgaaggtagggagggtagatgctctaccacattccttcagctgtattgagtgttccatttgcttatcaatttagagctcttctatggaaagagggtgaaaatctgttttgtgcctcttttttttgtatgtaaatgtgcaattatactggattttctcttgtaaaaaacactacaattcttttactgaagctcctaaactgccatttgcctgactccagcaattaattctgcggtgactcattgggcttccagtacttctgttgattagatatggtcccaaaagaaagagcaaaatggaaaatgcgatccatgtcaccaaatataattgctacaagtaacatgaaatacagctcaccaaaacactaaactttgctcttgagcaattatatagtttgatgtccttttaaaaaataagaaaagctaggttttatcatactaaaattttattttgtatatgatgtttggttttttaaacttactaacataaggatttcctttaatattccaaatacagttcttgaaacagtatccaacatgaaatcttatctccttgcttttagacttagtgctatttactatctaaagacttttctatggtaagccagggtacatcctatatactacagataccttaggtttcagtatttttatgcagcttcttcattgtgtcacattttgttttcatatctgtaacagagctctgctgaaattaaaaaaaaattatgattaataggattgtttcaccatttcagaaatcagagtagaaaacaaaatagctgccaggtgcagtggctcacacctgtaatcccagcactttgggaggccgaggtgggcagatcatgaggtcaggagtttgagaccagcctgaccaacatggtgaaaccctgtctctgctaaaaatacaaaaaggagccgggcatggtggcacatacctgtagtcccagtttctcaggaggcagaggttgcagtgagccaagatcacatcactgcactccagcctgggtgacagagcgagactccatctcaaaaaaaaaaggtaaccatagttctgaagtctttggccatgaatgttctttaatggtctaaaaagcggttttagtcatgctggtgccaaggagttctcctcacacctccatagcgtcaatgttgacctatttgatatatttttaagttgcagattatagatgcagaaagagaaaattgtcctttatgtgtgcatgagtatacatgcactgtgcctgtgatacaccaatgaaatattccaaaagacattgctcactcatagtctgaagttggtatttgttaacttttaaatctatatgtggtcagagtgtaagtggctggtttttattcttgtccaacaatgaaataacttaaagcagcatgggctaatggtggtattttttttaaccacatgttaatttgttgaaggaagaagtgaacataagatttttgataatcagcctaaagcttcagaaactggatcagtaagagctaaagatgaaatcctgggaaactttctcaggcactgtgcaggggagggaggggctggaccgccactgctgcgagggaagaatcagaagagctgcctgtggctggctgagaagggcaaggtggatggctcttcattcctggaaggctgaagcagccagaggcagtgattcacctctttaatgtctaatcagggagagatacaggagtgaggtgggttttattttcatttttgttttaacaagttcccactcttatcttaattctcaaaaatgatctggcctcatctatagaactggctgagttggggaatcatttaaaatttgggattttctgtacaagtcaagaacatatgtgaaagctaaaaaagtaatggaggcagggggactgctcccagctcccttctgccatttacacaaaatatttgaaaacatgtctgtaaataacgttgtggaactgagatattgagtatctaatcatgtaaaggaccctcagatgggacaggctattggacaactttcctggttcatgaagtggaatttcctttttgagtagtttcttgcatggtgtattccaataggtcaaacccagacctactgtttgcaaacaggccatctgtagagaatacaatctctggactgcccaggacttgttgcttgtctttcatttcattttaaatacaaagctcttcctttagcattcaccttgaagtcaaggtgttggctgccacatgccatgtcctcttaacaagataagaaaacagtggctatggagccctcacagctgggaagctgacattctatacttccatatgagaaaaaggacattggaaagttgctttatttctgggcagcgtagactaaagttacttttcacaaggctgatgcatccagcctcccccagttgtccaagttggtgcagacatgaattgatgaatggaaacactttcctttggcctagggccacctagaattggtctctggttatttgtaaaacaaaaagtagaacttgcttccattttcagatggactagggaatactataattagacagctttgcaatgaattaacaaatactgaaaagttggaaatgtgtatatactgaaatctcaccaacttttacctggggtggggtaccaggagaaagggaaccccctttctcctggtaaagggtaaggggggggataatgtttaccacaggtacgaaatagtcactttaacattgagacctctgcctcattgaattcaggttttttaagtacttgaaactcttcagattctccttattttagtttctttttacatttatgaagtagaaagcattgttttgtaaactgttttgaaaataaatagcctagtctcttatcctctttagcgtggattaaaggtgaagttctgcaaatgggagagtgttcacagtagatagctcagattgattgaacacatttgaggaagagactcctgcatgagataccagcatttttacaaatactttttatgtacattctttattttgtcattttgtcaaccctctccccaagcacatcttctttccttttactatgtctatgtagggaaaaaaacaaaacaaaaaattgcacttacgttacactcccaaaatgtgggtaatccgtgtctttcaaaaaacatttctgttttttgttttgttttggtcagtccattgcataagtgacaagtttgggtgcttgtggcacgtatgtatgaagcgggagggggatgagaattgcctgtccttcagtaggctgtaaaagtaatttacatgtaagtaaaaagggaaaatagaatagatgccaaagtcatttattcagtccttagttttcttatgtggcattactgcatctgctagttagtgagaaagcaccctcagcttttactgctcccctccctgcctgccaacacacttgatgtgtgcaaacagccctcaagtatctgtcagatgacctatataaggtattgaataaggtattcttgtcagtttagaaatggactggataaaacttacttggttgtcattattttatctcatttgtcctgttacatgccctatgttaagataattatattgccactaataatcaagatgctaaatgagtattacaactggctaatatcattttttatatacaagggtatgtgtatatttggaattgatatgagaaactcatttgtacccatttgagtgatattgcacaacaaacacagatacctacagactccgttttcattttctcgtgttctttatgataatgatctttgtagattggttatttctgtactttatctgtaataaactttgtagatcctgtgaaccattactttgcctaaatcacttgagacttgagtctttaataacaaagcatcaatattcactaaagtcaatctcttttgagtttctgtgacttggctagaagctcttgacactaagggattagtgttaattttccctgggggtgttccactagggcattactgtataatgacttgatgttgccacatagacttcaagatatataatattttgaggattttgttgattggcctatgttttattgcatagtgtgaaacgtgtaaagcttggttaacctgtatatagatagcttattgttgactagttatagtgtatttagggttgcctgtaatatttaagcttctttactgatgtgtgtgctggtaggaacatataatttttgtacattatatttactgagatgttgccttttttattttacaaatactttggaattccaatgtgttttttgcttccgtgaggattaatttggaaaggtttttaatgacattccactgatttcagattttgcttgagattgacttcaataaattgtcctgtatgttccaaa

SEQ ID NO: 17 - DNA, 25 bp, homo sapiens
ggtcgccgcggagaggcgtgcgaat

SEQ ID NO: 18 - DNA, 25 bp, homo sapiens
ccgtccctcggcctccccttcccat

SEQ ID NO: 19 - DNA, 25 bp, homo sapiens
ggccctcctcagtgccggccgccat

SEQ ID NO: 20 - DNA, 25 bp, homo sapiens
ggcagaggcgtccggcgcggggaaa

SEQ ID NO: 21 - DNA, 25 bp, homo sapiens
gcagaggcgtccggcgcggggaaaa

SEQ ID NO: 22 - DNA, 25 bp, homo sapiens
cgcggggaaaatctagcccggggat

SEQ ID NO: 23 - DNA, 25 bp, homo sapiens
gcggggaaaatctagcccggggatt

SEQ ID NO: 24 - DNA, 25 bp, homo sapiens
cggggaaaatctagcccggggattt

SEQ ID NO: 25 - DNA, 25 bp, homo sapiens
ggaaaatctagcccggggatttcat

SEQ ID NO: 26 - DNA, 25 bp, homo sapiens
ggatttcatgcggcctagctcggtt

SEQ ID NO: 27 - DNA, 25 bp, homo sapiens
cggccggggggttagaatggaacaa

SEQ ID NO: 28 - DNA, 25 bp, homo sapiens
ggccggggggttagaatggaacaaa

SEQ ID NO: 29 - DNA, 25 bp, homo sapiens
gggggttagaatggaacaaactgaa

SEQ ID NO: 30 - DNA, 25 bp, homo sapiens
ggcccgatgagagaaagggaaagtt

SEQ ID NO: 31 - DNA, 25 bp, homo sapiens
gcccgatgagagaaagggaaagtta

SEQ ID NO: 32 - DNA, 25 bp, homo sapiens
cccgatgagagaaagggaaagttaa

SEQ ID NO: 33 - DNA, 25 bp, homo sapiens
ggatgctggagcagaacaatggatt

SEQ ID NO: 34 - DNA, 25 bp, homo sapiens
gcaagggatcatgggaaacacaatt

SEQ ID NO: 35 - DNA, 25 bp, homo sapiens
ccaacatccgtcaggaggatgcctt

SEQ ID NO: 36 - DNA, 25 bp, homo sapiens
gctgaagatggtggccagacaccat

SEQ ID NO: 37 - DNA, 25 bp, homo sapiens
ggtggccagacaccatatgaagcta

SEQ ID NO: 38 - DNA, 25 bp, homo sapiens
ggccagacaccatatgaagctactt

SEQ ID NO: 39 - DNA, 25 bp, homo sapiens
gccagacaccatatgaagctacttt

SEQ ID NO: 40 - DNA, 25 bp, homo sapiens
cctccactcacaaatgggtatccat

SEQ ID NO: 41 - DNA, 25 bp, homo sapiens
ccactcacaaatgggtatccatcat

SEQ ID NO: 42 - DNA, 25 bp, homo sapiens
ggtatccatcatcaatcagtgtgta

SEQ ID NO: 43 - DNA, 25 bp, homo sapiens
ccatcatcaatcagtgtgtatgaaa

SEQ ID NO: 44 - DNA, 25 bp, homo sapiens
ccaaataccagtcatataatcagta

SEQ ID NO: 45 - DNA, 25 bp, homo sapiens
ccagtcatataatcagtatcctaat

SEQ ID NO: 46 - DNA, 25 bp, homo sapiens
gccaatggctttggtgcagttagaa

SEQ ID NO: 47 - DNA, 25 bp, homo sapiens
ccaatggctttggtgcagttagaaa

SEQ ID NO: 48 - DNA, 25 bp, homo sapiens
ggctttggtgcagttagaaacttta

SEQ ID NO: 49 - DNA, 25 bp, homo sapiens
ccccactgactattatcattcagaa

SEQ ID NO: 50 - DNA, 25 bp, homo sapiens
cccactgactattatcattcagaaa

SEQ ID NO: 51 - DNA, 25 bp, homo sapiens
ccactgactattatcattcagaaat

SEQ ID NO: 52 - DNA, 25 bp, homo sapiens
ccacatgaaattctggaaaaacctt

SEQ ID NO: 53 - DNA, 25 bp, homo sapiens
cctccacagccaccacctcctcctt

SEQ ID NO: 54 - DNA, 25 bp, homo sapiens
ccacctcctccttcggtaccacaaa

SEQ ID NO: 55 - DNA, 25 bp, homo sapiens
cggtaccacaaactgtgattccaaa

SEQ ID NO: 56 - DNA, 25 bp, homo sapiens
ccaaagaagactggctcacctgaaa

SEQ ID NO: 57 - DNA, 25 bp, homo sapiens
ggctcacctgaaattaaactaaaaa

SEQ ID NO: 58 - DNA, 25 bp, homo sapiens
gctcacctgaaattaaactaaaaat

SEQ ID NO: 59 - DNA, 25 bp, homo sapiens
cctttgtggagaccttttaaatgaa

SEQ ID NO: 60 - DNA, 25 bp, homo sapiens
cgaaatcaaagcatgaaagcagaaa

SEQ ID NO: 61 - DNA, 25 bp, homo sapiens
gcgcaagtcacacaaaatccccaaa

SEQ ID NO: 62 - DNA, 25 bp, homo sapiens
cgcaagtcacacaaaatccccaaat

SEQ ID NO: 63 - DNA, 25 bp, homo sapiens
gcaagtcacacaaaatccccaaatt

SEQ ID NO: 64 - DNA, 25 bp, homo sapiens
ccccaaattagaaccagaggaacaa

SEQ ID NO: 65 - DNA, 25 bp, homo sapiens
cccaaattagaaccagaggaacaaa

SEQ ID NO: 66 - DNA, 25 bp, homo sapiens
ccaaattagaaccagaggaacaaaa

SEQ ID NO: 67 - DNA, 25 bp, homo sapiens
ccaaatgagagggttgacactgtat

SEQ ID NO: 68 - DNA, 25 bp, homo sapiens
ggaagaaccagtactaaaagaggaa

SEQ ID NO: 69 - DNA, 25 bp, homo sapiens
ggaagccccagttcagccaatacta

SEQ ID NO: 70 - DNA, 25 bp, homo sapiens
gccccagttcagccaatactatctt

SEQ ID NO: 71 - DNA, 25 bp, homo sapiens
ccaatactatcttctgttccaacaa

SEQ ID NO: 72 - DNA, 25 bp, homo sapiens
cggaagtgtccactggtgttaagtt

SEQ ID NO: 73 - DNA, 25 bp, homo sapiens
ggaagtgtccactggtgttaagttt

SEQ ID NO: 74 - DNA, 25 bp, homo sapiens
ggaacctatccttggtggccttgta

SEQ ID NO: 75 - DNA, 25 bp, homo sapiens
ccttggtggccttgtatggtttcaa

SEQ ID NO: 76 - DNA, 25 bp, homo sapiens
ccccagcttgaggttcatactaaaa

SEQ ID NO: 77 - DNA, 25 bp, homo sapiens
cccagcttgaggttcatactaaaat

SEQ ID NO: 78 - DNA, 25 bp, homo sapiens
ccagcttgaggttcatactaaaatt

SEQ ID NO: 79 - DNA, 25 bp, homo sapiens
gcccgagaatatcatgtccagtttt

SEQ ID NO: 80 - DNA, 25 bp, homo sapiens
cccgagaatatcatgtccagttttt

SEQ ID NO: 81 - DNA, 25 bp, homo sapiens
ccgagaatatcatgtccagtttttt

SEQ ID NO: 82 - DNA, 25 bp, homo sapiens
cgagaatatcatgtccagtttttta

SEQ ID NO: 83 - DNA, 25 bp, homo sapiens
ccagccagagagggcgtgggttcat

SEQ ID NO: 84 - DNA, 25 bp, homo sapiens
gccagagagggcgtgggttcatgaa

SEQ ID NO: 85 - DNA, 25 bp, homo sapiens
ccagagagggcgtgggttcatgaaa

SEQ ID NO: 86 - DNA, 25 bp, homo sapiens
ggcgtgggttcatgaaaaacgggta

SEQ ID NO: 87 - DNA, 25 bp, homo sapiens
cgggtacgagagtataaaggtcata

SEQ ID NO: 88 - DNA, 25 bp, homo sapiens
gggtacgagagtataaaggtcataa

SEQ ID NO: 89 - DNA, 25 bp, homo sapiens
ggtacgagagtataaaggtcataaa

SEQ ID NO: 90 - DNA, 25 bp, homo sapiens
gccagcaatcactctgagaaacaaa

SEQ ID NO: 91 - DNA, 25 bp, homo sapiens
ccagcaatcactctgagaaacaaaa

SEQ ID NO: 92 - DNA, 25 bp, homo sapiens
gcaatcactctgagaaacaaaagat

SEQ ID NO: 93 - DNA, 25 bp, homo sapiens
ggcattgcccatgcagagaaagcat

SEQ ID NO: 94 - DNA, 25 bp, homo sapiens
gcattgcccatgcagagaaagcatt

SEQ ID NO: 95 - DNA, 25 bp, homo sapiens
gcccatgcagagaaagcattgaaaa

SEQ ID NO: 96 - DNA, 25 bp, homo sapiens
cccatgcagagaaagcattgaaaat

SEQ ID NO: 97 - DNA, 25 bp, homo sapiens
gcattgaaaatgactcgagaagaaa

SEQ ID NO: 98 - DNA, 25 bp, homo sapiens
cgagaagaaagaatagaacagtata

SEQ ID NO: 99 - DNA, 25 bp, homo sapiens
cctgaagaggctttatcccaagcaa

SEQ ID NO: 100 - DNA, 25 bp, homo sapiens
gcaaaaaagagtgttgcctccaaaa

SEQ ID NO: 101 - DNA, 25 bp, homo sapiens
gcctccaaaaccgaagttaaaaaaa

SEQ ID NO: 102 - DNA, 25 bp, homo sapiens
cccgacgaccaagatctgtgctgaa

SEQ ID NO: 103 - DNA, 25 bp, homo sapiens
ccgacgaccaagatctgtgctgaat

SEQ ID NO: 104 - DNA, 25 bp, homo sapiens
cgacgaccaagatctgtgctgaata

SEQ ID NO: 105 - DNA, 25 bp, homo sapiens
ggggaggtggcctcctcactctcaa

SEQ ID NO: 106 - DNA, 25 bp, homo sapiens
ggcctcctcactctcaagtactgaa

SEQ ID NO: 107 - DNA, 25 bp, homo sapiens
gcctcctcactctcaagtactgaaa

SEQ ID NO: 108 - DNA, 25 bp, homo sapiens
cctcctcactctcaagtactgaaat

SEQ ID NO: 109 - DNA, 25 bp, homo sapiens
ccagaggcggcacacaagtgcggaa

SEQ ID NO: 110 - DNA, 25 bp, homo sapiens
gcggcacacaagtgcggaagaggaa

SEQ ID NO: 111 - DNA, 25 bp, homo sapiens
ggaagaggaagagccaccgcctgtt

SEQ ID NO: 112 - DNA, 25 bp, homo sapiens
ggaagagccaccgcctgttaaaata

SEQ ID NO: 113 - DNA, 25 bp, homo sapiens
ccgcctgttaaaatagcctggaaaa

SEQ ID NO: 114 - DNA, 25 bp, homo sapiens
cctggaaaactgcggcagcaaggaa

SEQ ID NO: 115 - DNA, 25 bp, homo sapiens
gcagcaaggaaatccttaccagctt

SEQ ID NO: 116 - DNA, 25 bp, homo sapiens
cgatgcacaaagggagcctggattt

SEQ ID NO: 117 - DNA, 25 bp, homo sapiens
gggagcctggatttgcagaagtgta

SEQ ID NO: 118 - DNA, 25 bp, homo sapiens
ggagcctggatttgcagaagtgtaa

SEQ ID NO: 119 - DNA, 25 bp, homo sapiens
gcctggatttgcagaagtgtaacat

SEQ ID NO: 120 - DNA, 25 bp, homo sapiens
gcagaagtgtaacatgtctccagtt

SEQ ID NO: 121 - DNA, 25 bp, homo sapiens
ggggatgggaaatttatcgatcaat

SEQ ID NO: 122 - DNA, 25 bp, homo sapiens
gggatgggaaatttatcgatcaatt

SEQ ID NO: 123 - DNA, 25 bp, homo sapiens
ggatgggaaatttatcgatcaattt

SEQ ID NO: 124 - DNA, 25 bp, homo sapiens
gggaaatttatcgatcaatttgttt

SEQ ID NO: 125 - DNA, 25 bp, homo sapiens
ggaaatttatcgatcaatttgttta

SEQ ID NO: 126 - DNA, 25 bp, homo sapiens
gggaattggtaacaaaacagaaata

SEQ ID NO: 127 - DNA, 25 bp, homo sapiens
ggaattggtaacaaaacagaaataa

SEQ ID NO: 128 - DNA, 25 bp, homo sapiens
gggcaagacaggcttataatttcta

SEQ ID NO: 129 - DNA, 25 bp, homo sapiens
ccaaaccagagaaatgaaaagccaa

SEQ ID NO: 130 - DNA, 25 bp, homo sapiens
ccactgaggttgtgccaaagaagaa

SEQ ID NO: 131 - DNA, 25 bp, homo sapiens
ggttgtgccaaagaagaagatcaaa

SEQ ID NO: 132 - DNA, 25 bp, homo sapiens
cctcaggctacagtgaagactggat

SEQ ID NO: 133 - DNA, 25 bp, homo sapiens
gccagcgagatttcagattcctgta

SEQ ID NO: 134 - DNA, 25 bp, homo sapiens
ccagcgagatttcagattcctgtaa

SEQ ID NO: 135 - DNA, 25 bp, homo sapiens
gcctcaactgatgtagaaatgacta

SEQ ID NO: 136 - DNA, 25 bp, homo sapiens
cctgcaggtaggctttggaaagcaa

SEQ ID NO: 137 - DNA, 25 bp, homo sapiens
gcaggtaggctttggaaagcaagta

SEQ ID NO: 138 - DNA, 25 bp, homo sapiens
ggtaggctttggaaagcaagtagat

SEQ ID NO: 139 - DNA, 25 bp, homo sapiens
cgtttctgatgtgcagtccatggat

SEQ ID NO: 140 - DNA, 25 bp, homo sapiens
cgagaagaggcactggaatgagtaa

SEQ ID NO: 141 - DNA, 25 bp, homo sapiens
ggaatgagtaagaaggacactgtat

SEQ ID NO: 142 - DNA, 25 bp, homo sapiens
ggacactgtatgtcagatttgtgaa

SEQ ID NO: 143 - DNA, 25 bp, homo sapiens
ggagtgcctgggattggcatcactt

SEQ ID NO: 144 - DNA, 25 bp, homo sapiens
cctgggattggcatcacttcctgat

SEQ ID NO: 145 - DNA, 25 bp, homo sapiens
gcaagttcatctgcatggaatgtaa

SEQ ID NO: 146 - DNA, 25 bp, homo sapiens
gggcagcacccatgtttttcgtgta

SEQ ID NO: 147 - DNA, 25 bp, homo sapiens
ggcagcacccatgtttttcgtgtaa

SEQ ID NO: 148 - DNA, 25 bp, homo sapiens
gcagcacccatgtttttcgtgtaaa

SEQ ID NO: 149 - DNA, 25 bp, homo sapiens
ccgcaaattccccactgccatcttt

SEQ ID NO: 150 - DNA, 25 bp, homo sapiens
ccccactgccatctttgaatcaaaa

SEQ ID NO: 151 - DNA, 25 bp, homo sapiens
gctgctctgcctgctctatggagaa

SEQ ID NO: 152 - DNA, 25 bp, homo sapiens
gctctatggagaaagatatccacaa

SEQ ID NO: 153 - DNA, 25 bp, homo sapiens
gcaagtaaaggccgcatgatgagat

SEQ ID NO: 154 - DNA, 25 bp, homo sapiens
ggccgcatgatgagatgtttaagat

SEQ ID NO: 155 - DNA, 25 bp, homo sapiens
cctatcactctggagatgcttgcat

SEQ ID NO: 156 - DNA, 25 bp, homo sapiens
ggagatgcttgcattgcggccggaa

SEQ ID NO: 157 - DNA, 25 bp, homo sapiens
ccggaagcatgttagtatcctccta

SEQ ID NO: 158 - DNA, 25 bp, homo sapiens
cctcctacattctcatctgtagtaa

SEQ ID NO: 159 - DNA, 25 bp, homo sapiens
cggagcagtaattcttctgctgtaa

SEQ ID NO: 160 - DNA, 25 bp, homo sapiens
ggagcagtaattcttctgctgtaaa

SEQ ID NO: 161 - DNA, 25 bp, homo sapiens
cgtttgtgccagagggctgatagtt

SEQ ID NO: 162 - DNA, 25 bp, homo sapiens
ccccatgttcagttcatatgcctat

SEQ ID NO: 163 - DNA, 25 bp, homo sapiens
cccatgttcagttcatatgcctata

SEQ ID NO: 164 - DNA, 25 bp, homo sapiens
ccatgttcagttcatatgcctataa

SEQ ID NO: 165 - DNA, 25 bp, homo sapiens
cctataagtcccactacctactgaa

SEQ ID NO: 166 - DNA, 25 bp, homo sapiens
cccactacctactgaatgaatcaaa

SEQ ID NO: 167 - DNA, 25 bp, homo sapiens
ccactacctactgaatgaatcaaat

SEQ ID NO: 168 - DNA, 25 bp, homo sapiens
cgtgctgagttgatgaaattaccta

SEQ ID NO: 169 - DNA, 25 bp, homo sapiens
cctatgattccttcttcgtcagctt

SEQ ID NO: 170 - DNA, 25 bp, homo sapiens
ggtggaagattgctctgctgtgaat

SEQ ID NO: 171 - DNA, 25 bp, homo sapiens
gctgtgaatcgtgcccagcttcctt

SEQ ID NO: 172 - DNA, 25 bp, homo sapiens
gcttccttccacccggaatgcctaa

SEQ ID NO: 173 - DNA, 25 bp, homo sapiens
ccttccacccggaatgcctaagcat

SEQ ID NO: 174 - DNA, 25 bp, homo sapiens
ccacccggaatgcctaagcatagaa

SEQ ID NO: 175 - DNA, 25 bp, homo sapiens
gccagaaggctgctggaattgtaat

SEQ ID NO: 176 - DNA, 25 bp, homo sapiens
ggctgctggaattgtaatgactgta

SEQ ID NO: 177 - DNA, 25 bp, homo sapiens
gctgctggaattgtaatgactgtaa

SEQ ID NO: 178 - DNA, 25 bp, homo sapiens
gcaagaaactacattacaagcagat

SEQ ID NO: 179 - DNA, 25 bp, homo sapiens
gcaaccccaggtctgtgccactgaa

SEQ ID NO: 180 - DNA, 25 bp, homo sapiens
gccactgaacatccagggccttaaa

SEQ ID NO: 181 - DNA, 25 bp, homo sapiens
gccttaaacatgacttgggggactt

SEQ ID NO: 182 - DNA, 25 bp, homo sapiens
gggggacttccctgtattcttcttt

SEQ ID NO: 183 - DNA, 25 bp, homo sapiens
ccctgtattcttctttggttctcat

SEQ ID NO: 184 - DNA, 25 bp, homo sapiens
ccagggcagagtgttcccttatgtt

SEQ ID NO: 185 - DNA, 25 bp, homo sapiens
gggcagagtgttcccttatgttgaa

SEQ ID NO: 186 - DNA, 25 bp, homo sapiens
gctttgctgaagggcagactagtat

SEQ ID NO: 187 - DNA, 25 bp, homo sapiens
ggcagactagtattaacaagacctt

SEQ ID NO: 188 - DNA, 25 bp, homo sapiens
gcactggaagaagctgcaaaacgtt

SEQ ID NO: 189 - DNA, 25 bp, homo sapiens
cgtttccaggaattgaaagcacaaa

SEQ ID NO: 190 - DNA, 25 bp, homo sapiens
ccaggaattgaaagcacaaagagaa

SEQ ID NO: 191 - DNA, 25 bp, homo sapiens
gccctagagattgaaaaaaactcaa

SEQ ID NO: 192 - DNA, 25 bp, homo sapiens
cctccctacaaacacatcaaagcta

SEQ ID NO: 193 - DNA, 25 bp, homo sapiens
cctacaaacacatcaaagctaacaa

SEQ ID NO: 194 - DNA, 25 bp, homo sapiens
ccaggttgctgacctgtcagagatt

SEQ ID NO: 195 - DNA, 25 bp, homo sapiens
ccgctgtaactgcaagccagctgat

SEQ ID NO: 196 - DNA, 25 bp, homo sapiens
cctgaacagaatgttgcagtatgaa

SEQ ID NO: 197 - DNA, 25 bp, homo sapiens
cccgcaggtgtgcccagctggagat

SEQ ID NO: 198 - DNA, 25 bp, homo sapiens
gcccagctggagatcgttgtcagaa

SEQ ID NO: 199 - DNA, 25 bp, homo sapiens
cgttgtcagaaccagtgctttacaa

SEQ ID NO: 200 - DNA, 25 bp, homo sapiens
ggagaggctggggcctcaggaccaa

SEQ ID NO: 201 - DNA, 25 bp, homo sapiens
ggggcctcaggaccaaaaggagcat

SEQ ID NO: 202 - DNA, 25 bp, homo sapiens
gggcctcaggaccaaaaggagcatt

SEQ ID NO: 203 - DNA, 25 bp, homo sapiens
ggcctcaggaccaaaaggagcatta

SEQ ID NO: 204 - DNA, 25 bp, homo sapiens
gcctcaggaccaaaaggagcattaa

SEQ ID NO: 205 - DNA, 25 bp, homo sapiens
gcattaagaagggtgaatttgtaaa

SEQ ID NO: 206 - DNA, 25 bp, homo sapiens
cgtcggtgaattaattgatgaagaa

SEQ ID NO: 207 - DNA, 25 bp, homo sapiens
cggtgaattaattgatgaagaagaa

SEQ ID NO: 208 - DNA, 25 bp, homo sapiens
ggtgaattaattgatgaagaagaat

SEQ ID NO: 209 - DNA, 25 bp, homo sapiens
cgagcccacgagaacagtgtaacta

SEQ ID NO: 210 - DNA, 25 bp, homo sapiens
gcccacgagaacagtgtaactaatt

SEQ ID NO: 211 - DNA, 25 bp, homo sapiens
cccacgagaacagtgtaactaattt

SEQ ID NO: 212 - DNA, 25 bp, homo sapiens
ccacgagaacagtgtaactaatttt

SEQ ID NO: 213 - DNA, 25 bp, homo sapiens
cgagaacagtgtaactaatttttat

SEQ ID NO: 214 - DNA, 25 bp, homo sapiens
ccgtataattgatgccggcccaaaa

SEQ ID NO: 215 - DNA, 25 bp, homo sapiens
gcccaaaaggaaattattctcgctt

SEQ ID NO: 216 - DNA, 25 bp, homo sapiens
ccacagttgtaatcccaactgtgaa

SEQ ID NO: 217 - DNA, 25 bp, homo sapiens
cctgcagggatggagttaacattta

SEQ ID NO: 218 - DNA, 25 bp, homo sapiens
gcagggatggagttaacatttaatt

SEQ ID NO: 219 - DNA, 25 bp, homo sapiens
gggatggagttaacatttaattata

SEQ ID NO: 220 - DNA, 25 bp, homo sapiens
ggatggagttaacatttaattataa

SEQ ID NO: 221 - DNA, 25 bp, homo sapiens
ggagttaacatttaattataaccta

SEQ ID NO: 222 - DNA, 25 bp, homo sapiens
cggagtgccactgtggagcagataa

SEQ ID NO: 223 - DNA, 25 bp, homo sapiens
cggccaaagtcggcatgtgcgtcaa

SEQ ID NO: 224 - DNA, 25 bp, homo sapiens
ccaaagtcggcatgtgcgtcaacaa

SEQ ID NO: 225 - DNA, 25 bp, homo sapiens
gcgtcaacaaatgaagagaaggcaa

SEQ ID NO: 226 - DNA, 25 bp, homo sapiens
cgtcaacaaatgaagagaaggcaaa

SEQ ID NO: 227 - DNA, 25 bp, homo sapiens
gctaagttaaaacagaagagacgaa

SEQ ID NO: 228 - DNA, 25 bp, homo sapiens
gcagatgcatgaagattactgtttt

SEQ ID NO: 229 - DNA, 25 bp, homo sapiens
ggagagctggtcatgtgtgacaaaa

SEQ ID NO: 230 - DNA, 25 bp, homo sapiens
gccttaacctgactcagccaccata

SEQ ID NO: 231 - DNA, 25 bp, homo sapiens
ccttaacctgactcagccaccatat

SEQ ID NO: 232 - DNA, 25 bp, homo sapiens
ggagtgtccgtggcatcagtgcgat

SEQ ID NO: 233 - DNA, 25 bp, homo sapiens
cgatgagtgcagcagtgcagctgtt

SEQ ID NO: 234 - DNA, 25 bp, homo sapiens
ggccctggttccctctgcactggaa

SEQ ID NO: 235 - DNA, 25 bp, homo sapiens
ggaaggccgcctctgctgctcggaa

SEQ ID NO: 236 - DNA, 25 bp, homo sapiens
ccccatggctcctgtgtcaccagaa

SEQ ID NO: 237 - DNA, 25 bp, homo sapiens
cccatggctcctgtgtcaccagaat

SEQ ID NO: 238 - DNA, 25 bp, homo sapiens
ccatggctcctgtgtcaccagaata

SEQ ID NO: 239 - DNA, 25 bp, homo sapiens
ccagaatactggagcaagataaaat

SEQ ID NO: 240 - DNA, 25 bp, homo sapiens
ggaatcacaagatcatggagaagaa

SEQ ID NO: 241 - DNA, 25 bp, homo sapiens
cccctcctttctatttaagtgaaaa

SEQ ID NO: 242 - DNA, 25 bp, homo sapiens
ccctcctttctatttaagtgaaaaa

SEQ ID NO: 243 - DNA, 25 bp, homo sapiens
cctcctttctatttaagtgaaaaaa

SEQ ID NO: 244 - DNA, 25 bp, homo sapiens
gcctttgccatcggaactgccttat

SEQ ID NO: 245 - DNA, 25 bp, homo sapiens
cctttgccatcggaactgccttatt

SEQ ID NO: 246 - DNA, 25 bp, homo sapiens
cggaactgccttattaaagcaaaaa

SEQ ID NO: 247 - DNA, 25 bp, homo sapiens
ggaactgccttattaaagcaaaaat

SEQ ID NO: 248 - DNA, 25 bp, homo sapiens
gccttattaaagcaaaaatgggaaa

SEQ ID NO: 249 - DNA, 25 bp, homo sapiens
gggaaaccagttcatgcaggcagaa

SEQ ID NO: 250 - DNA, 25 bp, homo sapiens
ccagttcatgcaggcagaagcagtt

SEQ ID NO: 251 - DNA, 25 bp, homo sapiens
ggtggtgtctggtttttgtttgatt

SEQ ID NO: 252 - DNA, 25 bp, homo sapiens
ggtttttgtttgatttggttggttt

SEQ ID NO: 253 - DNA, 25 bp, homo sapiens
gggattcttttgtggagggttaaat

SEQ ID NO: 254 - DNA, 25 bp, homo sapiens
ggattcttttgtggagggttaaatt

SEQ ID NO: 255 - DNA, 25 bp, homo sapiens
ggagggttaaattcccttggtcttt

SEQ ID NO: 256 - DNA, 25 bp, homo sapiens
ggttaaattcccttggtcttttctt

SEQ ID NO: 257 - DNA, 25 bp, homo sapiens
cccttggtcttttcttgccttttat

SEQ ID NO: 258 - DNA, 25 bp, homo sapiens
ccttggtcttttcttgccttttatt

SEQ ID NO: 259 - DNA, 25 bp, homo sapiens
gccttttattgtgcttcagtgccat

SEQ ID NO: 260 - DNA, 25 bp, homo sapiens
ccttttattgtgcttcagtgccatt

SEQ ID NO: 261 - DNA, 25 bp, homo sapiens
gcttcagtgccattgcagcttgaaa

SEQ ID NO: 262 - DNA, 25 bp, homo sapiens
gctgttaaaataagaacaaagagaa

SEQ ID NO: 263 - DNA, 25 bp, homo sapiens
ggttgtataaagcaaaagtagccat

SEQ ID NO: 264 - DNA, 25 bp, homo sapiens
gcaaaagtagccatcattcctttat

SEQ ID NO: 265 - DNA, 25 bp, homo sapiens
ccatcattcctttatttattttcat

SEQ ID NO: 266 - DNA, 25 bp, homo sapiens
ggaatttcaagaagtgtagttcaat

SEQ ID NO: 267 - DNA, 25 bp, homo sapiens
ggaatctgagaaagccctctaggaa

SEQ ID NO: 268 - DNA, 25 bp, homo sapiens
ggaaagggtatgataagctttatat

SEQ ID NO: 269 - DNA, 25 bp, homo sapiens
ccttcaaaatgcttttcataggctt

SEQ ID NO: 270 - DNA, 25 bp, homo sapiens
cggtggattttacctctgtggtctt

SEQ ID NO: 271 - DNA, 25 bp, homo sapiens
ggtggattttacctctgtggtctta

SEQ ID NO: 272 - DNA, 25 bp, homo sapiens
ggtcttaaacaaggtgggctttgta

SEQ ID NO: 273 - DNA, 25 bp, homo sapiens
ggtgggctttgtactatggtaactt

SEQ ID NO: 274 - DNA, 25 bp, homo sapiens
ggtaactttacctagaagggtatat

SEQ ID NO: 275 - DNA, 25 bp, homo sapiens
ggtgtgtttccttggtggaaggaaa

SEQ ID NO: 276 - DNA, 25 bp, homo sapiens
ccttggtggaaggaaaaaaaggaat

SEQ ID NO: 277 - DNA, 25 bp, homo sapiens
ggtggaaggaaaaaaaggaatggat

SEQ ID NO: 278 - DNA, 25 bp, homo sapiens
ggaatggatgtgctcactgattttt

SEQ ID NO: 279 - DNA, 25 bp, homo sapiens
cgttttccattgacatggatggttt

SEQ ID NO: 280 - DNA, 25 bp, homo sapiens
ccattgacatggatggtttgggttt

SEQ ID NO: 281 - DNA, 25 bp, homo sapiens
ggatgaagtgcgtttctgtggatat

SEQ ID NO: 282 - DNA, 25 bp, homo sapiens
gcatagttggaatggtcactcctat

SEQ ID NO: 283 - DNA, 25 bp, homo sapiens
cctgacactgattaaggcagctaaa

SEQ ID NO: 284 - DNA, 25 bp, homo sapiens
ggcagctaaagggggatgaaggcat

SEQ ID NO: 285 - DNA, 25 bp, homo sapiens
gcagctaaagggggatgaaggcata

SEQ ID NO: 286 - DNA, 25 bp, homo sapiens
gggggatgaaggcataaaccaaaat

SEQ ID NO: 287 - DNA, 25 bp, homo sapiens
ggggatgaaggcataaaccaaaatt

SEQ ID NO: 288 - DNA, 25 bp, homo sapiens
gggatgaaggcataaaccaaaattt

SEQ ID NO: 289 - DNA, 25 bp, homo sapiens
ggcataaaccaaaatttggatcata

SEQ ID NO: 290 - DNA, 25 bp, homo sapiens
gcataaaccaaaatttggatcatat

SEQ ID NO: 291 - DNA, 25 bp, homo sapiens
gcaccattttctgtccaattttatt

SEQ ID NO: 292 - DNA, 25 bp, homo sapiens
ccattttctgtccaattttattgta

SEQ ID NO: 293 - DNA, 25 bp, homo sapiens
ccctggtcttaattggagagaatta

SEQ ID NO: 294 - DNA, 25 bp, homo sapiens
ggagagaattacaggcattttattt

SEQ ID NO: 295 - DNA, 25 bp, homo sapiens
cctctcctactcaaacatttctttt

SEQ ID NO: 296 - DNA, 25 bp, homo sapiens
cctactcaaacatttcttttgtcat

SEQ ID NO: 297 - DNA, 25 bp, homo sapiens
ccattcagtcatgtttcctggtaaa

SEQ ID NO: 298 - DNA, 25 bp, homo sapiens
cctggtaaaaccacacaagtgagtt

SEQ ID NO: 299 - DNA, 25 bp, homo sapiens
ccacacaagtgagtttgaattgtat

SEQ ID NO: 300 - DNA, 25 bp, homo sapiens
cctaatttggggggtgaaagtagta

SEQ ID NO: 301 - DNA, 25 bp, homo sapiens
ggggcatccccaggttacatgtaaa

SEQ ID NO: 302 - DNA, 25 bp, homo sapiens
gcatccccaggttacatgtaaagaa

SEQ ID NO: 303 - DNA, 25 bp, homo sapiens
ccaggttacatgtaaagaatttcta

SEQ ID NO: 304 - DNA, 25 bp, homo sapiens
ggttacatgtaaagaatttctatta

SEQ ID NO: 305 - DNA, 25 bp, homo sapiens
gcataagagccacacaaacacactt

SEQ ID NO: 306 - DNA, 25 bp, homo sapiens
ggaagcacttggagaaaactcccaa

SEQ ID NO: 307 - DNA, 25 bp, homo sapiens
gcacttggagaaaactcccaagaaa

SEQ ID NO: 308 - DNA, 25 bp, homo sapiens
cccaagaaagtcagtgttagcaaaa

SEQ ID NO: 309 - DNA, 25 bp, homo sapiens
ccaagaaagtcagtgttagcaaaaa

SEQ ID NO: 310 - DNA, 25 bp, homo sapiens
gcaaaaatggttgatggtgatgctt

SEQ ID NO: 311 - DNA, 25 bp, homo sapiens
ggttgatggtgatgcttgactctta

SEQ ID NO: 312 - DNA, 25 bp, homo sapiens
gcttgactcttatagatgcgagtat

SEQ ID NO: 313 - DNA, 25 bp, homo sapiens
gctcaccaaactcacttgcttttaa

SEQ ID NO: 314 - DNA, 25 bp, homo sapiens
ccaaactcacttgcttttaatgctt

SEQ ID NO: 315 - DNA, 25 bp, homo sapiens
gcttttaatgcttaggaatatgtat

SEQ ID NO: 316 - DNA, 25 bp, homo sapiens
gcttaggaatatgtatattccagta

SEQ ID NO: 317 - DNA, 25 bp, homo sapiens
ccagtattccttgtatgaaaataat

SEQ ID NO: 318 - DNA, 25 bp, homo sapiens
ggttagcttgaatgaatgttaactt

SEQ ID NO: 319 - DNA, 25 bp, homo sapiens
gctggtacctggtagcatttatttt

SEQ ID NO: 320 - DNA, 25 bp, homo sapiens
cctggtagcatttattttccctcaa

SEQ ID NO: 321 - DNA, 25 bp, homo sapiens
ggtagcatttattttccctcaagta

SEQ ID NO: 322 - DNA, 25 bp, homo sapiens
gcatttattttccctcaagtaccta

SEQ ID NO: 323 - DNA, 25 bp, homo sapiens
ccttatgaaggtagggagggtagat

SEQ ID NO: 324 - DNA, 25 bp, homo sapiens
gggagggtagatgctctaccacatt

SEQ ID NO: 325 - DNA, 25 bp, homo sapiens
gggtagatgctctaccacattcctt

SEQ ID NO: 326 - DNA, 25 bp, homo sapiens
gctctaccacattccttcagctgta

SEQ ID NO: 327 - DNA, 25 bp, homo sapiens
ccttcagctgtattgagtgttccat

SEQ ID NO: 328 - DNA, 25 bp, homo sapiens
gctgtattgagtgttccatttgctt

SEQ ID NO: 329 - DNA, 25 bp, homo sapiens
gcttatcaatttagagctcttctat

SEQ ID NO: 330 - DNA, 25 bp, homo sapiens
gctcttctatggaaagagggtgaaa

SEQ ID NO: 331 - DNA, 25 bp, homo sapiens
ggtgaaaatctgttttgtgcctctt

SEQ ID NO: 332 - DNA, 25 bp, homo sapiens
gccatttgcctgactccagcaatta

SEQ ID NO: 333 - DNA, 25 bp, homo sapiens
ccatttgcctgactccagcaattaa

SEQ ID NO: 334 - DNA, 25 bp, homo sapiens
gcaattaattctgcggtgactcatt

SEQ ID NO: 335 - DNA, 25 bp, homo sapiens
gcggtgactcattgggcttccagta

SEQ ID NO: 336 - DNA, 25 bp, homo sapiens
gggcttccagtacttctgttgatta

SEQ ID NO: 337 - DNA, 25 bp, homo sapiens
cccaaaagaaagagcaaaatggaaa

SEQ ID NO: 338 - DNA, 25 bp, homo sapiens
ccaaaagaaagagcaaaatggaaaa

SEQ ID NO: 339 - DNA, 25 bp, homo sapiens
ggaaaatgcgatccatgtcaccaaa

SEQ ID NO: 340 - DNA, 25 bp, homo sapiens
gcgatccatgtcaccaaatataatt

SEQ ID NO: 341 - DNA, 25 bp, homo sapiens
gcaattatatagtttgatgtccttt

SEQ ID NO: 342 - DNA, 25 bp, homo sapiens
gctaggttttatcatactaaaattt

SEQ ID NO: 343 - DNA, 25 bp, homo sapiens
ggttttatcatactaaaattttatt

SEQ ID NO: 344 - DNA, 25 bp, homo sapiens
ccaaatacagttcttgaaacagtat

SEQ ID NO: 345 - DNA, 25 bp, homo sapiens
ccttgcttttagacttagtgctatt

SEQ ID NO: 346 - DNA, 25 bp, homo sapiens
ggtaagccagggtacatcctatata

SEQ ID NO: 347 - DNA, 25 bp, homo sapiens
gggtacatcctatatactacagata

SEQ ID NO: 348 - DNA, 25 bp, homo sapiens
gcagcttcttcattgtgtcacattt

SEQ ID NO: 349 - DNA, 25 bp, homo sapiens
gctctgctgaaattaaaaaaaaatt

SEQ ID NO: 350 - DNA, 25 bp, homo sapiens
gctgaaattaaaaaaaaattatgat

SEQ ID NO: 351 - DNA, 25 bp, homo sapiens
ccatttcagaaatcagagtagaaaa

SEQ ID NO: 352 - DNA, 25 bp, homo sapiens
ccaggtgcagtggctcacacctgta

SEQ ID NO: 353 - DNA, 25 bp, homo sapiens
gctcacacctgtaatcccagcactt

SEQ ID NO: 354 - DNA, 25 bp, homo sapiens
gggcagatcatgaggtcaggagttt

SEQ ID NO: 355 - DNA, 25 bp, homo sapiens
ccgggcatggtggcacatacctgta

SEQ ID NO: 356 - DNA, 25 bp, homo sapiens
cccagtttctcaggaggcagaggtt

SEQ ID NO: 357 - DNA, 25 bp, homo sapiens
ggaggcagaggttgcagtgagccaa

SEQ ID NO: 358 - DNA, 25 bp, homo sapiens
ggcagaggttgcagtgagccaagat

SEQ ID NO: 359 - DNA, 25 bp, homo sapiens
cctgggtgacagagcgagactccat

SEQ ID NO: 360 - DNA, 25 bp, homo sapiens
gcgagactccatctcaaaaaaaaaa

SEQ ID NO: 361 - DNA, 25 bp, homo sapiens
ccatagttctgaagtctttggccat

SEQ ID NO: 362 - DNA, 25 bp, homo sapiens
gccatgaatgttctttaatggtcta

SEQ ID NO: 363 - DNA, 25 bp, homo sapiens
ccatgaatgttctttaatggtctaa

SEQ ID NO: 364 - DNA, 25 bp, homo sapiens
gcggttttagtcatgctggtgccaa

SEQ ID NO: 365 - DNA, 25 bp, homo sapiens
cctccatagcgtcaatgttgaccta

SEQ ID NO: 366 - DNA, 25 bp, homo sapiens
ccatagcgtcaatgttgacctattt

SEQ ID NO: 367 - DNA, 25 bp, homo sapiens
gcgtcaatgttgacctatttgatat

SEQ ID NO: 368 - DNA, 25 bp, homo sapiens
cgtcaatgttgacctatttgatata

SEQ ID NO: 369 - DNA, 25 bp, homo sapiens
gcagattatagatgcagaaagagaa

SEQ ID NO: 370 - DNA, 25 bp, homo sapiens
gcagaaagagaaaattgtcctttat

SEQ ID NO: 371 - DNA, 25 bp, homo sapiens
cctttatgtgtgcatgagtatacat

SEQ ID NO: 372 - DNA, 25 bp, homo sapiens
gcctgtgatacaccaatgaaatatt

SEQ ID NO: 373 - DNA, 25 bp, homo sapiens
ccaatgaaatattccaaaagacatt

SEQ ID NO: 374 - DNA, 25 bp, homo sapiens
gctcactcatagtctgaagttggta

SEQ ID NO: 375 - DNA, 25 bp, homo sapiens
ggtatttgttaacttttaaatctat

SEQ ID NO: 376 - DNA, 25 bp, homo sapiens
ggtcagagtgtaagtggctggtttt

SEQ ID NO: 377 - DNA, 25 bp, homo sapiens
gctggtttttattcttgtccaacaa

SEQ ID NO: 378 - DNA, 25 bp, homo sapiens
gcagcatgggctaatggtggtattt

SEQ ID NO: 379 - DNA, 25 bp, homo sapiens
gcatgggctaatggtggtatttttt

SEQ ID NO: 380 - DNA, 25 bp, homo sapiens
gggctaatggtggtattttttttaa

SEQ ID NO: 381 - DNA, 25 bp, homo sapiens
ggtattttttttaaccacatgttaa

SEQ ID NO: 382 - DNA, 25 bp, homo sapiens
ggatcagtaagagctaaagatgaaa

SEQ ID NO: 383 - DNA, 25 bp, homo sapiens
gcctgtggctggctgagaagggcaa

SEQ ID NO: 384 - DNA, 25 bp, homo sapiens
gctggctgagaagggcaaggtggat

SEQ ID NO: 385 - DNA, 25 bp, homo sapiens
ggtggatggctcttcattcctggaa

SEQ ID NO: 386 - DNA, 25 bp, homo sapiens
ggctcttcattcctggaaggctgaa

SEQ ID NO: 387 - DNA, 25 bp, homo sapiens
ggctgaagcagccagaggcagtgat

SEQ ID NO: 388 - DNA, 25 bp, homo sapiens
gctgaagcagccagaggcagtgatt

SEQ ID NO: 389 - DNA, 25 bp, homo sapiens
gccagaggcagtgattcacctcttt

SEQ ID NO: 390 - DNA, 25 bp, homo sapiens
ccagaggcagtgattcacctcttta

SEQ ID NO: 391 - DNA, 25 bp, homo sapiens
ggagtgaggtgggttttattttcat

SEQ ID NO: 392 - DNA, 25 bp, homo sapiens
ggtgggttttattttcatttttgtt

SEQ ID NO: 393 - DNA, 25 bp, homo sapiens
gggttttattttcatttttgtttta

SEQ ID NO: 394 - DNA, 25 bp, homo sapiens
ggttttattttcatttttgttttaa

SEQ ID NO: 395 - DNA, 25 bp, homo sapiens
cccactcttatcttaattctcaaaa

SEQ ID NO: 396 - DNA, 25 bp, homo sapiens
ccactcttatcttaattctcaaaaa

SEQ ID NO: 397 - DNA, 25 bp, homo sapiens
cctcatctatagaactggctgagtt

SEQ ID NO: 398 - DNA, 25 bp, homo sapiens
ggctgagttggggaatcatttaaaa

SEQ ID NO: 399 - DNA, 25 bp, homo sapiens
gctgagttggggaatcatttaaaat

SEQ ID NO: 400 - DNA, 25 bp, homo sapiens
ggggaatcatttaaaatttgggatt

SEQ ID NO: 401 - DNA, 25 bp, homo sapiens
gggaatcatttaaaatttgggattt

SEQ ID NO: 402 - DNA, 25 bp, homo sapiens
ggaatcatttaaaatttgggatttt

SEQ ID NO: 403 - DNA, 25 bp, homo sapiens
gctcccagctcccttctgccattta

SEQ ID NO: 404 - DNA, 25 bp, homo sapiens
gctcccttctgccatttacacaaaa

SEQ ID NO: 405 - DNA, 25 bp, homo sapiens
cccttctgccatttacacaaaatat

SEQ ID NO: 406 - DNA, 25 bp, homo sapiens
ccttctgccatttacacaaaatatt

SEQ ID NO: 407 - DNA, 25 bp, homo sapiens
gccatttacacaaaatatttgaaaa

SEQ ID NO: 408 - DNA, 25 bp, homo sapiens
cgttgtggaactgagatattgagta

SEQ ID NO: 409 - DNA, 25 bp, homo sapiens
ggaactgagatattgagtatctaat

SEQ ID NO: 410 - DNA, 25 bp, homo sapiens
ggaccctcagatgggacaggctatt

SEQ ID NO: 411 - DNA, 25 bp, homo sapiens
ggttcatgaagtggaatttcctttt

SEQ ID NO: 412 - DNA, 25 bp, homo sapiens
gcatggtgtattccaataggtcaaa

SEQ ID NO: 413 - DNA, 25 bp, homo sapiens
gcaaacaggccatctgtagagaata

SEQ ID NO: 414 - DNA, 25 bp, homo sapiens
ccaggacttgttgcttgtctttcat

SEQ ID NO: 415 - DNA, 25 bp, homo sapiens
gcttgtctttcatttcattttaaat

SEQ ID NO: 416 - DNA, 25 bp, homo sapiens
cctttagcattcaccttgaagtcaa

SEQ ID NO: 417 - DNA, 25 bp, homo sapiens
gcattcaccttgaagtcaaggtgtt

SEQ ID NO: 418 - DNA, 25 bp, homo sapiens
ggctgccacatgccatgtcctctta

SEQ ID NO: 419 - DNA, 25 bp, homo sapiens
gctgccacatgccatgtcctcttaa

SEQ ID NO: 420 - DNA, 25 bp, homo sapiens
gccacatgccatgtcctcttaacaa

SEQ ID NO: 421 - DNA, 25 bp, homo sapiens
ccatgtcctcttaacaagataagaa

SEQ ID NO: 422 - DNA, 25 bp, homo sapiens
gctatggagccctcacagctgggaa

SEQ ID NO: 423 - DNA, 25 bp, homo sapiens
gccctcacagctgggaagctgacat

SEQ ID NO: 424 - DNA, 25 bp, homo sapiens
ccctcacagctgggaagctgacatt

SEQ ID NO: 425 - DNA, 25 bp, homo sapiens
gctgggaagctgacattctatactt

SEQ ID NO: 426 - DNA, 25 bp, homo sapiens
ggaagctgacattctatacttccat

SEQ ID NO: 427 - DNA, 25 bp, homo sapiens
ccatatgagaaaaaggacattggaa

SEQ ID NO: 428 - DNA, 25 bp, homo sapiens
gggcagcgtagactaaagttacttt

SEQ ID NO: 429 - DNA, 25 bp, homo sapiens
ggcagcgtagactaaagttactttt

SEQ ID NO: 430 - DNA, 25 bp, homo sapiens
cgtagactaaagttacttttcacaa

SEQ ID NO: 431 - DNA, 25 bp, homo sapiens
ccagcctcccccagttgtccaagtt

SEQ ID NO: 432 - DNA, 25 bp, homo sapiens
gcagacatgaattgatgaatggaaa

SEQ ID NO: 433 - DNA, 25 bp, homo sapiens
cctttggcctagggccacctagaat

SEQ ID NO: 434 - DNA, 25 bp, homo sapiens
ccattttcagatggactagggaata

SEQ ID NO: 435 - DNA, 25 bp, homo sapiens
gggaatactataattagacagcttt

SEQ ID NO: 436 - DNA, 25 bp, homo sapiens
gcaatgaattaacaaatactgaaaa

SEQ ID NO: 437 - DNA, 25 bp, homo sapiens
gggtggggtaccaggagaaagggaa

SEQ ID NO: 438 - DNA, 25 bp, homo sapiens
ccccctttctcctggtaaagggtaa

SEQ ID NO: 439 - DNA, 25 bp, homo sapiens
cctggtaaagggtaaggggggggat

SEQ ID NO: 440 - DNA, 25 bp, homo sapiens
ggtaaagggtaaggggggggataat

SEQ ID NO: 441 - DNA, 25 bp, homo sapiens
ggggggataatgtttaccacaggta

SEQ ID NO: 442 - DNA, 25 bp, homo sapiens
ggataatgtttaccacaggtacgaa

SEQ ID NO: 443 - DNA, 25 bp, homo sapiens
ccacaggtacgaaatagtcacttta

SEQ ID NO: 444 - DNA, 25 bp, homo sapiens
ggtacgaaatagtcactttaacatt

SEQ ID NO: 445 - DNA, 25 bp, homo sapiens
cctctgcctcattgaattcaggttt

SEQ ID NO: 446 - DNA, 25 bp, homo sapiens
gcctcattgaattcaggttttttaa

SEQ ID NO: 447 - DNA, 25 bp, homo sapiens
ggttttttaagtacttgaaactctt

SEQ ID NO: 448 - DNA, 25 bp, homo sapiens
ccttattttagtttctttttacatt

SEQ ID NO: 449 - DNA, 25 bp, homo sapiens
gcattgttttgtaaactgttttgaa

SEQ ID NO: 450 - DNA, 25 bp, homo sapiens
ggattaaaggtgaagttctgcaaat

SEQ ID NO: 451 - DNA, 25 bp, homo sapiens
cctgcatgagataccagcattttta

SEQ ID NO: 452 - DNA, 25 bp, homo sapiens
gcatgagataccagcatttttacaa

SEQ ID NO: 453 - DNA, 25 bp, homo sapiens
ccagcatttttacaaatacttttta

SEQ ID NO: 454 - DNA, 25 bp, homo sapiens
ccctctccccaagcacatcttcttt

SEQ ID NO: 455 - DNA, 25 bp, homo sapiens
ccccaagcacatcttctttcctttt

SEQ ID NO: 456 - DNA, 25 bp, homo sapiens
cccaagcacatcttctttcctttta

SEQ ID NO: 457 - DNA, 25 bp, homo sapiens
ccttttactatgtctatgtagggaa

SEQ ID NO: 458 - DNA, 25 bp, homo sapiens
cgttacactcccaaaatgtgggtaa

SEQ ID NO: 459 - DNA, 25 bp, homo sapiens
ccaaaatgtgggtaatccgtgtctt

SEQ ID NO: 460 - DNA, 25 bp, homo sapiens
ggtcagtccattgcataagtgacaa

SEQ ID NO: 461 - DNA, 25 bp, homo sapiens
gcataagtgacaagtttgggtgctt

SEQ ID NO: 462 - DNA, 25 bp, homo sapiens
ggtgcttgtggcacgtatgtatgaa

SEQ ID NO: 463 - DNA, 25 bp, homo sapiens
cgtatgtatgaagcgggagggggat

SEQ ID NO: 464 - DNA, 25 bp, homo sapiens
gcctgtccttcagtaggctgtaaaa

SEQ ID NO: 465 - DNA, 25 bp, homo sapiens
ccttcagtaggctgtaaaagtaatt

SEQ ID NO: 466 - DNA, 25 bp, homo sapiens
ggctgtaaaagtaatttacatgtaa

SEQ ID NO: 467 - DNA, 25 bp, homo sapiens
gccaaagtcatttattcagtcctta

SEQ ID NO: 468 - DNA, 25 bp, homo sapiens
cccctccctgcctgccaacacactt

SEQ ID NO: 469 - DNA, 25 bp, homo sapiens
cctcaagtatctgtcagatgaccta

SEQ ID NO: 470 - DNA, 25 bp, homo sapiens
cctatataaggtattgaataaggta

SEQ ID NO: 471 - DNA, 25 bp, homo sapiens
ggataaaacttacttggttgtcatt

SEQ ID NO: 472 - DNA, 25 bp, homo sapiens
cctgttacatgccctatgttaagat

SEQ ID NO: 473 - DNA, 25 bp, homo sapiens
gccactaataatcaagatgctaaat

SEQ ID NO: 474 - DNA, 25 bp, homo sapiens
gctaaatgagtattacaactggcta

SEQ ID NO: 475 - DNA, 25 bp, homo sapiens
ggctaatatcattttttatatacaa

SEQ ID NO: 476 - DNA, 25 bp, homo sapiens
gggtatgtgtatatttggaattgat

SEQ ID NO: 477 - DNA, 25 bp, homo sapiens
ggtatgtgtatatttggaattgata

SEQ ID NO: 478 - DNA, 25 bp, homo sapiens
ccatttgagtgatattgcacaacaa

SEQ ID NO: 479 - DNA, 25 bp, homo sapiens
ccgttttcattttctcgtgttcttt

SEQ ID NO: 480 - DNA, 25 bp, homo sapiens
cgttttcattttctcgtgttcttta

SEQ ID NO: 481 - DNA, 25 bp, homo sapiens
cgtgttctttatgataatgatcttt

SEQ ID NO: 482 - DNA, 25 bp, homo sapiens
ggttatttctgtactttatctgtaa

SEQ ID NO: 483 - DNA, 25 bp, homo sapiens
cctgtgaaccattactttgcctaaa

SEQ ID NO: 484 - DNA, 25 bp, homo sapiens
ccctgggggtgttccactagggcat

SEQ ID NO: 485 - DNA, 25 bp, homo sapiens
cctgggggtgttccactagggcatt

SEQ ID NO: 486 - DNA, 25 bp, homo sapiens
ggtgttccactagggcattactgta

SEQ ID NO: 487 - DNA, 25 bp, homo sapiens
gggcattactgtataatgacttgat

SEQ ID NO: 488 - DNA, 25 bp, homo sapiens
gccacatagacttcaagatatataa

SEQ ID NO: 489 - DNA, 25 bp, homo sapiens
ccacatagacttcaagatatataat

SEQ ID NO: 490 - DNA, 25 bp, homo sapiens
ggattttgttgattggcctatgttt

SEQ ID NO: 491 - DNA, 25 bp, homo sapiens
cctatgttttattgcatagtgtgaa

SEQ ID NO: 492 - DNA, 25 bp, homo sapiens
gcatagtgtgaaacgtgtaaagctt

SEQ ID NO: 493 - DNA, 25 bp, homo sapiens
cgtgtaaagcttggttaacctgtat

SEQ ID NO: 494 - DNA, 25 bp, homo sapiens
ggttaacctgtatatagatagctta

SEQ ID NO: 495 - DNA, 25 bp, homo sapiens
ggaacatataatttttgtacattat

SEQ ID NO: 496 - DNA, 25 bp, homo sapiens
gccttttttattttacaaatacttt

SEQ ID NO: 497 - DNA, 25 bp, homo sapiens
gcttccgtgaggattaatttggaaa

SEQ ID NO: 498 - DNA, 25 bp, homo sapiens
ccgtgaggattaatttggaaaggtt

SEQ ID NO: 499 - DNA, 25 bp, homo sapiens
cgtgaggattaatttggaaaggttt

SEQ ID NO: 500 - DNA, 25 bp, homo sapiens
ggattaatttggaaaggtttttaat

SEQ ID NO: 501 - DNA, 25 bp, homo sapiens
ggtttttaatgacattccactgatt

SEQ ID NO: 502 - DNA, 19 bp, homo sapiens
ccgcggagaggcgtgcgaa

SEQ ID NO: 503 - DNA, 19 bp, homo sapiens
cgcggagaggcgtgcgaat

SEQ ID NO: 504 - DNA, 19 bp, homo sapiens
gcggagaggcgtgcgaatt

SEQ ID NO: 505 - DNA, 19 bp, homo sapiens
cgtccctcggcctcccctt

SEQ ID NO: 506 - DNA, 19 bp, homo sapiens
cctcccgagccttcctctt

SEQ ID NO: 507 - DNA, 19 bp, homo sapiens
cctcagtgccggccgccat

SEQ ID NO: 508 - DNA, 19 bp, homo sapiens
ggcgtccggcgcggggaaa

SEQ ID NO: 509 - DNA, 19 bp, homo sapiens
gcgtccggcgcggggaaaa

SEQ ID NO: 510 - DNA, 19 bp, homo sapiens
cgtccggcgcggggaaaat

SEQ ID NO: 511 - DNA, 19 bp, homo sapiens
ccggcgcggggaaaatcta

SEQ ID NO: 512 - DNA, 19 bp, homo sapiens
ggggatttcatgcggccta

SEQ ID NO: 513 - DNA, 19 bp, homo sapiens
ccggggggttagaatggaa

SEQ ID NO: 514 - DNA, 19 bp, homo sapiens
gggggttagaatggaacaa

SEQ ID NO: 515 - DNA, 19 bp, homo sapiens
ggggttagaatggaacaaa

SEQ ID NO: 516 - DNA, 19 bp, homo sapiens
cccgatgagagaaagggaa

SEQ ID NO: 517 - DNA, 19 bp, homo sapiens
ccgatgagagaaagggaaa

SEQ ID NO: 518 - DNA, 19 bp, homo sapiens
ggatgctggagcagaacaa

SEQ ID NO: 519 - DNA, 19 bp, homo sapiens
ggagcagaacaatggattt

SEQ ID NO: 520 - DNA, 19 bp, homo sapiens
gggatcatgggaaacacaa

SEQ ID NO: 521 - DNA, 19 bp, homo sapiens
ggatcatgggaaacacaat

SEQ ID NO: 522 - DNA, 19 bp, homo sapiens
gggaaacacaattcagcaa

SEQ ID NO: 523 - DNA, 19 bp, homo sapiens
gcaaccacctcaactcatt

SEQ ID NO: 524 - DNA, 19 bp, homo sapiens
ccgtcaggaggatgccttt

SEQ ID NO: 525 - DNA, 19 bp, homo sapiens
ggctttcagtacccagcta

SEQ ID NO: 526 - DNA, 19 bp, homo sapiens
cccagctacaacagaagat

SEQ ID NO: 527 - DNA, 19 bp, homo sapiens
ccaaataccagtcatataa

SEQ ID NO: 528 - DNA, 19 bp, homo sapiens
ccagtcatataatcagtat

SEQ ID NO: 529 - DNA, 19 bp, homo sapiens
ggctttggtgcagttagaa

SEQ ID NO: 530 - DNA, 19 bp, homo sapiens
gctttggtgcagttagaaa

SEQ ID NO: 531 - DNA, 19 bp, homo sapiens
ggtgcagttagaaacttta

SEQ ID NO: 532 - DNA, 19 bp, homo sapiens
ccccactgactattatcat

SEQ ID NO: 533 - DNA, 19 bp, homo sapiens
cccactgactattatcatt

SEQ ID NO: 534 - DNA, 19 bp, homo sapiens
ccacatgaaattctggaaa

SEQ ID NO: 535 - DNA, 19 bp, homo sapiens
cctccttcggtaccacaaa

SEQ ID NO: 536 - DNA, 19 bp, homo sapiens
cggtaccacaaactgtgat

SEQ ID NO: 537 - DNA, 19 bp, homo sapiens
ggtaccacaaactgtgatt

SEQ ID NO: 538 - DNA, 19 bp, homo sapiens
ccacaaactgtgattccaa

SEQ ID NO: 539 - DNA, 19 bp, homo sapiens
cctgaaattaaactaaaaa

SEQ ID NO: 540 - DNA, 19 bp, homo sapiens
ccctttgtggagacctttt

SEQ ID NO: 541 - DNA, 19 bp, homo sapiens
cctttgtggagacctttta

SEQ ID NO: 542 - DNA, 19 bp, homo sapiens
ggcaagtgagcacacgaaa

SEQ ID NO: 543 - DNA, 19 bp, homo sapiens
gcaagtgagcacacgaaat

SEQ ID NO: 544 - DNA, 19 bp, homo sapiens
gcacacgaaatcaaagcat

SEQ ID NO: 545 - DNA, 19 bp, homo sapiens
gcatgaaagcagaaaagaa

SEQ ID NO: 546 - DNA, 19 bp, homo sapiens
gcagaaaagaaaagaggaa

SEQ ID NO: 547 - DNA, 19 bp, homo sapiens
ggaacaaaatagaccaaat

SEQ ID NO: 548 - DNA, 19 bp, homo sapiens
ggttgacactgtatcagaa

SEQ ID NO: 549 - DNA, 19 bp, homo sapiens
gggaagaaccagtactaaa

SEQ ID NO: 550 - DNA, 19 bp, homo sapiens
ggaagaaccagtactaaaa

SEQ ID NO: 551 - DNA, 19 bp, homo sapiens
cccagttcagccaatacta

SEQ ID NO: 552 - DNA, 19 bp, homo sapiens
ccagttcagccaatactat

SEQ ID NO: 553 - DNA, 19 bp, homo sapiens
gccaatactatcttctgtt

SEQ ID NO: 554 - DNA, 19 bp, homo sapiens
ggaagtgtccactggtgtt

SEQ ID NO: 555 - DNA, 19 bp, homo sapiens
gcgatcttgtgtggtccaa

SEQ ID NO: 556 - DNA, 19 bp, homo sapiens
ggtccaaggtgggaaccta

SEQ ID NO: 557 - DNA, 19 bp, homo sapiens
ccccagcttgaggttcata

SEQ ID NO: 558 - DNA, 19 bp, homo sapiens
gcttgaggttcatactaaa

SEQ ID NO: 559 - DNA, 19 bp, homo sapiens
gccagagagggcgtgggtt

SEQ ID NO: 560 - DNA, 19 bp, homo sapiens
gggcgtgggttcatgaaaa

SEQ ID NO: 561 - DNA, 19 bp, homo sapiens
ggcgtgggttcatgaaaaa

SEQ ID NO: 562 - DNA, 19 bp, homo sapiens
ggttcatgaaaaacgggta

SEQ ID NO: 563 - DNA, 19 bp, homo sapiens
cgagagtataaaggtcata

SEQ ID NO: 564 - DNA, 19 bp, homo sapiens
ggctgaggcaaccaaacaa

SEQ ID NO: 565 - DNA, 19 bp, homo sapiens
ccagcaatcactctgagaa

SEQ ID NO: 566 - DNA, 19 bp, homo sapiens
ggatattggcattgcccat

SEQ ID NO: 567 - DNA, 19 bp, homo sapiens
gcattgcccatgcagagaa

SEQ ID NO: 568 - DNA, 19 bp, homo sapiens
gcccatgcagagaaagcat

SEQ ID NO: 569 - DNA, 19 bp, homo sapiens
cccatgcagagaaagcatt

SEQ ID NO: 570 - DNA, 19 bp, homo sapiens
gcagagaaagcattgaaaa

SEQ ID NO: 571 - DNA, 19 bp, homo sapiens
ggctttatcccaagcaaaa

SEQ ID NO: 572 - DNA, 19 bp, homo sapiens
gctttatcccaagcaaaaa

SEQ ID NO: 573 - DNA, 19 bp, homo sapiens
ccaagcaaaaaagagtgtt

SEQ ID NO: 574 - DNA, 19 bp, homo sapiens
gcctccaaaaccgaagtta

SEQ ID NO: 575 - DNA, 19 bp, homo sapiens
cctccaaaaccgaagttaa

SEQ ID NO: 576 - DNA, 19 bp, homo sapiens
ccaaaaccgaagttaaaaa

SEQ ID NO: 577 - DNA, 19 bp, homo sapiens
ccaagatctgtgctgaata

SEQ ID NO: 578 - DNA, 19 bp, homo sapiens
gctgaatactcagccagaa

SEQ ID NO: 579 - DNA, 19 bp, homo sapiens
gcctcctcactctcaagta

SEQ ID NO: 580 - DNA, 19 bp, homo sapiens
gcggcacacaagtgcggaa

SEQ ID NO: 581 - DNA, 19 bp, homo sapiens
ggaagagccaccgcctgtt

SEQ ID NO: 582 - DNA, 19 bp, homo sapiens
gccaccgcctgttaaaata

SEQ ID NO: 583 - DNA, 19 bp, homo sapiens
cggcagcaaggaaatcctt

SEQ ID NO: 584 - DNA, 19 bp, homo sapiens
ggcagcaaggaaatcctta

SEQ ID NO: 585 - DNA, 19 bp, homo sapiens
gcacaaagggagcctggat

SEQ ID NO: 586 - DNA, 19 bp, homo sapiens
ggagcctggatttgcagaa

SEQ ID NO: 587 - DNA, 19 bp, homo sapiens
gctacaggggatgggaaat

SEQ ID NO: 588 - DNA, 19 bp, homo sapiens
ggatgggaaatttatcgat

SEQ ID NO: 589 - DNA, 19 bp, homo sapiens
gggaaatttatcgatcaat

SEQ ID NO: 590 - DNA, 19 bp, homo sapiens
ggaaatttatcgatcaatt

SEQ ID NO: 591 - DNA, 19 bp, homo sapiens
ggtaacaaaacagaaataa

SEQ ID NO: 592 - DNA, 19 bp, homo sapiens
gggggcaagacaggcttat

SEQ ID NO: 593 - DNA, 19 bp, homo sapiens
ggggcaagacaggcttata

SEQ ID NO: 594 - DNA, 19 bp, homo sapiens
gggcaagacaggcttataa

SEQ ID NO: 595 - DNA, 19 bp, homo sapiens
ggcaagacaggcttataat

SEQ ID NO: 596 - DNA, 19 bp, homo sapiens
gcaagacaggcttataatt

SEQ ID NO: 597 - DNA, 19 bp, homo sapiens
ccaaaccagagaaatgaaa

SEQ ID NO: 598 - DNA, 19 bp, homo sapiens
ccaacgcagagtgtatcat

SEQ ID NO: 599 - DNA, 19 bp, homo sapiens
cctgaagcaacatctggtt

SEQ ID NO: 600 - DNA, 19 bp, homo sapiens
gcaacagagaagatcaatt

SEQ ID NO: 601 - DNA, 19 bp, homo sapiens
cgttctgaatcagagaaat

SEQ ID NO: 602 - DNA, 19 bp, homo sapiens
ccactgaggttgtgccaaa

SEQ ID NO: 603 - DNA, 19 bp, homo sapiens
gccaaagaagaagatcaaa

SEQ ID NO: 604 - DNA, 19 bp, homo sapiens
ccaaagaagaagatcaaaa

SEQ ID NO: 605 - DNA, 19 bp, homo sapiens
ggagcaggttgaaacagtt

SEQ ID NO: 606 - DNA, 19 bp, homo sapiens
gctacagtgaagactggat

SEQ ID NO: 607 - DNA, 19 bp, homo sapiens
gccagcgagatttcagatt

SEQ ID NO: 608 - DNA, 19 bp, homo sapiens
cctgtaaacctctaaagaa

SEQ ID NO: 609 - DNA, 19 bp, homo sapiens
cgcctcaactgatgtagaa

SEQ ID NO: 610 - DNA, 19 bp, homo sapiens
gcctcaactgatgtagaaa

SEQ ID NO: 611 - DNA, 19 bp, homo sapiens
cctcaactgatgtagaaat

SEQ ID NO: 612 - DNA, 19 bp, homo sapiens
ggtaggctttggaaagcaa

SEQ ID NO: 613 - DNA, 19 bp, homo sapiens
cccttcagctactgcagat

SEQ ID NO: 614 - DNA, 19 bp, homo sapiens
cgagaagaggcactggaat

SEQ ID NO: 615 - DNA, 19 bp, homo sapiens
gcactggaatgagtaagaa

SEQ ID NO: 616 - DNA, 19 bp, homo sapiens
ggacactgtatgtcagatt

SEQ ID NO: 617 - DNA, 19 bp, homo sapiens
cctgggattggcatcactt

SEQ ID NO: 618 - DNA, 19 bp, homo sapiens
gggcagcacccatgttttt

SEQ ID NO: 619 - DNA, 19 bp, homo sapiens
cccatgtttttcgtgtaaa

SEQ ID NO: 620 - DNA, 19 bp, homo sapiens
cgtgtaaagtgtctggtaa

SEQ ID NO: 621 - DNA, 19 bp, homo sapiens
ggtaaagatgtgaagcgtt

SEQ ID NO: 622 - DNA, 19 bp, homo sapiens
cgttgttctgttggtgctt

SEQ ID NO: 623 - DNA, 19 bp, homo sapiens
ggtgcttgtgggaaatttt

SEQ ID NO: 624 - DNA, 19 bp, homo sapiens
gcaaattccccactgccat

SEQ ID NO: 625 - DNA, 19 bp, homo sapiens
ccccactgccatctttgaa

SEQ ID NO: 626 - DNA, 19 bp, homo sapiens
cccactgccatctttgaat

SEQ ID NO: 627 - DNA, 19 bp, homo sapiens
gctgctctgcctgctctat

SEQ ID NO: 628 - DNA, 19 bp, homo sapiens
gctctatggagaaagatat

SEQ ID NO: 629 - DNA, 19 bp, homo sapiens
ggagaaagatatccacaaa

SEQ ID NO: 630 - DNA, 19 bp, homo sapiens
ggccgcatgatgagatgtt

SEQ ID NO: 631 - DNA, 19 bp, homo sapiens
gccgcatgatgagatgttt

SEQ ID NO: 632 - DNA, 19 bp, homo sapiens
ccgcatgatgagatgttta

SEQ ID NO: 633 - DNA, 19 bp, homo sapiens
cgcatgatgagatgtttaa

SEQ ID NO: 634 - DNA, 19 bp, homo sapiens
gcttgcattgcggccggaa

SEQ ID NO: 635 - DNA, 19 bp, homo sapiens
gcattgcggccggaagcat

SEQ ID NO: 636 - DNA, 19 bp, homo sapiens
ggccggaagcatgttagta

SEQ ID NO: 637 - DNA, 19 bp, homo sapiens
gccggaagcatgttagtat

SEQ ID NO: 638 - DNA, 19 bp, homo sapiens
gcatgttagtatcctccta

SEQ ID NO: 639 - DNA, 19 bp, homo sapiens
gctgtaaatgtaggctttt

SEQ ID NO: 640 - DNA, 19 bp, homo sapiens
gctgatagttcaggaccat

SEQ ID NO: 641 - DNA, 19 bp, homo sapiens
ccccatgttcagttcatat

SEQ ID NO: 642 - DNA, 19 bp, homo sapiens
ccactacctactgaatgaa

SEQ ID NO: 643 - DNA, 19 bp, homo sapiens
cctactgaatgaatcaaat

SEQ ID NO: 644 - DNA, 19 bp, homo sapiens
cgtgctgagttgatgaaat

SEQ ID NO: 645 - DNA, 19 bp, homo sapiens
cgtcagcttccaaaaagaa

SEQ ID NO: 646 - DNA, 19 bp, homo sapiens
ccaaaaagaaatgtgagaa

SEQ ID NO: 647 - DNA, 19 bp, homo sapiens
gcttccttccacccggaat

SEQ ID NO: 648 - DNA, 19 bp, homo sapiens
cccggaatgcctaagcata

SEQ ID NO: 649 - DNA, 19 bp, homo sapiens
ggaatgcctaagcatagaa

SEQ ID NO: 650 - DNA, 19 bp, homo sapiens
gccagaaggctgctggaat

SEQ ID NO: 651 - DNA, 19 bp, homo sapiens
ccagaaggctgctggaatt

SEQ ID NO: 652 - DNA, 19 bp, homo sapiens
ggaattgtaatgactgtaa

SEQ ID NO: 653 - DNA, 19 bp, homo sapiens
gctggcaagaaactacatt

SEQ ID NO: 654 - DNA, 19 bp, homo sapiens
gcaagaaactacattacaa

SEQ ID NO: 655 - DNA, 19 bp, homo sapiens
gcagattgtttgggtcaaa

SEQ ID NO: 656 - DNA, 19 bp, homo sapiens
gggtcaaattgggaaatta

SEQ ID NO: 657 - DNA, 19 bp, homo sapiens
ggccagcagagatctgcaa

SEQ ID NO: 658 - DNA, 19 bp, homo sapiens
ccaggtctgtgccactgaa

SEQ ID NO: 659 - DNA, 19 bp, homo sapiens
ggtctgtgccactgaacat

SEQ ID NO: 660 - DNA, 19 bp, homo sapiens
gggacttccctgtattctt

SEQ ID NO: 661 - DNA, 19 bp, homo sapiens
cctgtattcttctttggtt

SEQ ID NO: 662 - DNA, 19 bp, homo sapiens
gggcagagtgttcccttat

SEQ ID NO: 663 - DNA, 19 bp, homo sapiens
gctgaagggcagactagta

SEQ ID NO: 664 - DNA, 19 bp, homo sapiens
ggcagactagtattaacaa

SEQ ID NO: 665 - DNA, 19 bp, homo sapiens
gcactggaagaagctgcaa

SEQ ID NO: 666 - DNA, 19 bp, homo sapiens
gcaaaacgtttccaggaat

SEQ ID NO: 667 - DNA, 19 bp, homo sapiens
ccaggaattgaaagcacaa

SEQ ID NO: 668 - DNA, 19 bp, homo sapiens
gccctagagattgaaaaaa

SEQ ID NO: 669 - DNA, 19 bp, homo sapiens
ccctagagattgaaaaaaa

SEQ ID NO: 670 - DNA, 19 bp, homo sapiens
cccctccctacaaacacat

SEQ ID NO: 671 - DNA, 19 bp, homo sapiens
gctaacaaagtaataggaa

SEQ ID NO: 672 - DNA, 19 bp, homo sapiens
gcaagccagctgatgaaaa

SEQ ID NO: 673 - DNA, 19 bp, homo sapiens
ccagctgatgaaaaccctt

SEQ ID NO: 674 - DNA, 19 bp, homo sapiens
cggagtgcctgaacagaat

SEQ ID NO: 675 - DNA, 19 bp, homo sapiens
ggtgtgcccagctggagat

SEQ ID NO: 676 - DNA, 19 bp, homo sapiens
gctttacaaagagactata

SEQ ID NO: 677 - DNA, 19 bp, homo sapiens
gctggggcctcaggaccaa

SEQ ID NO: 678 - DNA, 19 bp, homo sapiens
ggaccaaaaggagcattaa

SEQ ID NO: 679 - DNA, 19 bp, homo sapiens
ccaaaaggagcattaagaa

SEQ ID NO: 680 - DNA, 19 bp, homo sapiens
gcattaagaagggtgaatt

SEQ ID NO: 681 - DNA, 19 bp, homo sapiens
gggtgaatttgtaaatgaa

SEQ ID NO: 682 - DNA, 19 bp, homo sapiens
ggtgaatttgtaaatgaat

SEQ ID NO: 683 - DNA, 19 bp, homo sapiens
cgtcggtgaattaattgat

SEQ ID NO: 684 - DNA, 19 bp, homo sapiens
cggtgaattaattgatgaa

SEQ ID NO: 685 - DNA, 19 bp, homo sapiens
gcccacgagaacagtgtaa

SEQ ID NO: 686 - DNA, 19 bp, homo sapiens
cgagaacagtgtaactaat

SEQ ID NO: 687 - DNA, 19 bp, homo sapiens
gccggcccaaaaggaaatt

SEQ ID NO: 688 - DNA, 19 bp, homo sapiens
ccggcccaaaaggaaatta

SEQ ID NO: 689 - DNA, 19 bp, homo sapiens
cggcccaaaaggaaattat

SEQ ID NO: 690 - DNA, 19 bp, homo sapiens
ggcccaaaaggaaattatt

SEQ ID NO: 691 - DNA, 19 bp, homo sapiens
cgcttcatgaaccacagtt

SEQ ID NO: 692 - DNA, 19 bp, homo sapiens
cccaactgtgaaacacaaa

SEQ ID NO: 693 - DNA, 19 bp, homo sapiens
ccaactgtgaaacacaaaa

SEQ ID NO: 694 - DNA, 19 bp, homo sapiens
cctgcagggatggagttaa

SEQ ID NO: 695 - DNA, 19 bp, homo sapiens
gcagggatggagttaacat

SEQ ID NO: 696 - DNA, 19 bp, homo sapiens
gggatggagttaacattta

SEQ ID NO: 697 - DNA, 19 bp, homo sapiens
ggatggagttaacatttaa

SEQ ID NO: 698 - DNA, 19 bp, homo sapiens
ggagttaacatttaattat

SEQ ID NO: 699 - DNA, 19 bp, homo sapiens
gccactgtggagcagataa

SEQ ID NO: 700 - DNA, 19 bp, homo sapiens
gcagataactgcagtggtt

SEQ ID NO: 701 - DNA, 19 bp, homo sapiens
cggcatgtgcgtcaacaaa

SEQ ID NO: 702 - DNA, 19 bp, homo sapiens
ggcatgtgcgtcaacaaat

SEQ ID NO: 703 - DNA, 19 bp, homo sapiens
cgtcaacaaatgaagagaa

SEQ ID NO: 704 - DNA, 19 bp, homo sapiens
ggcaaaaaatgctaagtta

SEQ ID NO: 705 - DNA, 19 bp, homo sapiens
gcaaaaaatgctaagttaa

SEQ ID NO: 706 - DNA, 19 bp, homo sapiens
gcatgaagattactgtttt

SEQ ID NO: 707 - DNA, 19 bp, homo sapiens
gctggtcatgtgtgacaaa

SEQ ID NO: 708 - DNA, 19 bp, homo sapiens
ggtcatgtgtgacaaaaaa

SEQ ID NO: 709 - DNA, 19 bp, homo sapiens
cctgactcagccaccatat

SEQ ID NO: 710 - DNA, 19 bp, homo sapiens
gcagtgcagctgtttcctt

SEQ ID NO: 711 - DNA, 19 bp, homo sapiens
gctgtttccttctgtgaat

SEQ ID NO: 712 - DNA, 19 bp, homo sapiens
ggttccctctgcactggaa

SEQ ID NO: 713 - DNA, 19 bp, homo sapiens
ccgcctctgctgctcggaa

SEQ ID NO: 714 - DNA, 19 bp, homo sapiens
cctctgctgctcggaacat

SEQ ID NO: 715 - DNA, 19 bp, homo sapiens
gctcggaacatgaccccat

SEQ ID NO: 716 - DNA, 19 bp, homo sapiens
ggctcctgtgtcaccagaa

SEQ ID NO: 717 - DNA, 19 bp, homo sapiens
gctcctgtgtcaccagaat

SEQ ID NO: 718 - DNA, 19 bp, homo sapiens
ggagcaagataaaatgtaa

SEQ ID NO: 719 - DNA, 19 bp, homo sapiens
ggagaagaagtaaaagaat

SEQ ID NO: 720 - DNA, 19 bp, homo sapiens
ggtgtcccctcctttctat

SEQ ID NO: 721 - DNA, 19 bp, homo sapiens
cctttctatttaagtgaaa

SEQ ID NO: 722 - DNA, 19 bp, homo sapiens
gcaaatagatcatgcattt

SEQ ID NO: 723 - DNA, 19 bp, homo sapiens
gccatcggaactgccttat

SEQ ID NO: 724 - DNA, 19 bp, homo sapiens
ccatcggaactgccttatt

SEQ ID NO: 725 - DNA, 19 bp, homo sapiens
gccttattaaagcaaaaat

SEQ ID NO: 726 - DNA, 19 bp, homo sapiens
ccagttcatgcaggcagaa

SEQ ID NO: 727 - DNA, 19 bp, homo sapiens
ggttggtttgggattcttt

SEQ ID NO: 728 - DNA, 19 bp, homo sapiens
ggtcttttcttgcctttta

SEQ ID NO: 729 - DNA, 19 bp, homo sapiens
gccattgcagcttgaaaaa

SEQ ID NO: 730 - DNA, 19 bp, homo sapiens
gcttgaaaaagaaatgttt

SEQ ID NO: 731 - DNA, 19 bp, homo sapiens
gctgttaaaataagaacaa

SEQ ID NO: 732 - DNA, 19 bp, homo sapiens
ccttggttgtataaagcaa

SEQ ID NO: 733 - DNA, 19 bp, homo sapiens
gccatcattcctttattta

SEQ ID NO: 734 - DNA, 19 bp, homo sapiens
ccatcattcctttatttat

SEQ ID NO: 735 - DNA, 19 bp, homo sapiens
cctttatttattttcattt

SEQ ID NO: 736 - DNA, 19 bp, homo sapiens
gccctctaggaaagggtat

SEQ ID NO: 737 - DNA, 19 bp, homo sapiens
gggtatgataagctttata

SEQ ID NO: 738 - DNA, 19 bp, homo sapiens
ggtatgataagctttatat

SEQ ID NO: 739 - DNA, 19 bp, homo sapiens
gcagtaggtaggctcactt

SEQ ID NO: 740 - DNA, 19 bp, homo sapiens
ccttcaaaatgcttttcat

SEQ ID NO: 741 - DNA, 19 bp, homo sapiens
ggcttagagaagggctcta

SEQ ID NO: 742 - DNA, 19 bp, homo sapiens
gcttagagaagggctctat

SEQ ID NO: 743 - DNA, 19 bp, homo sapiens
gggctctatggaagtatta

SEQ ID NO: 744 - DNA, 19 bp, homo sapiens
ggctctatggaagtattaa

SEQ ID NO: 745 - DNA, 19 bp, homo sapiens
gctctatggaagtattaaa

SEQ ID NO: 746 - DNA, 19 bp, homo sapiens
cctctgtggtcttaaacaa

SEQ ID NO: 747 - DNA, 19 bp, homo sapiens
gggctttgtactatggtaa

SEQ ID NO: 748 - DNA, 19 bp, homo sapiens
ccttggtggaaggaaaaaa

SEQ ID NO: 749 - DNA, 19 bp, homo sapiens
ggatgtgctcactgatttt

SEQ ID NO: 750 - DNA, 19 bp, homo sapiens
ccattgacatggatggttt

SEQ ID NO: 751 - DNA, 19 bp, homo sapiens
ggtttgggttttggatgaa

SEQ ID NO: 752 - DNA, 19 bp, homo sapiens
gcgtttctgtggatatgaa

SEQ ID NO: 753 - DNA, 19 bp, homo sapiens
ggaatggtcactcctattt

SEQ ID NO: 754 - DNA, 19 bp, homo sapiens
gcagctaaagggggatgaa

SEQ ID NO: 755 - DNA, 19 bp, homo sapiens
ggatgaaggcataaaccaa

SEQ ID NO: 756 - DNA, 19 bp, homo sapiens
ccaaaatttggatcatata

SEQ ID NO: 757 - DNA, 19 bp, homo sapiens
ggatcatataagcaccatt

SEQ ID NO: 758 - DNA, 19 bp, homo sapiens
gcaccattttctgtccaat

SEQ ID NO: 759 - DNA, 19 bp, homo sapiens
ccattttctgtccaatttt

SEQ ID NO: 760 - DNA, 19 bp, homo sapiens
ggtcttaattggagagaat

SEQ ID NO: 761 - DNA, 19 bp, homo sapiens
ggagagaattacaggcatt

SEQ ID NO: 762 - DNA, 19 bp, homo sapiens
cctctcctactcaaacatt

SEQ ID NO: 763 - DNA, 19 bp, homo sapiens
cctactcaaacatttcttt

SEQ ID NO: 764 - DNA, 19 bp, homo sapiens
ccacacaagtgagtttgaa

SEQ ID NO: 765 - DNA, 19 bp, homo sapiens
cctaatttggggggtgaaa

SEQ ID NO: 766 - DNA, 19 bp, homo sapiens
gggcatccccaggttacat

SEQ ID NO: 767 - DNA, 19 bp, homo sapiens
ccaggttacatgtaaagaa

SEQ ID NO: 768 - DNA, 19 bp, homo sapiens
ggttacatgtaaagaattt

SEQ ID NO: 769 - DNA, 19 bp, homo sapiens
ggggaagaacttctacttt

SEQ ID NO: 770 - DNA, 19 bp, homo sapiens
gggaagaacttctactttt

SEQ ID NO: 771 - DNA, 19 bp, homo sapiens
ggaagaacttctactttta

SEQ ID NO: 772 - DNA, 19 bp, homo sapiens
ccacacaaacacacttcat

SEQ ID NO: 773 - DNA, 19 bp, homo sapiens
ggagaaaactcccaagaaa

SEQ ID NO: 774 - DNA, 19 bp, homo sapiens
cccaagaaagtcagtgtta

SEQ ID NO: 775 - DNA, 19 bp, homo sapiens
ggtgatgcttgactcttat

SEQ ID NO: 776 - DNA, 19 bp, homo sapiens
ccaaactcacttgctttta

SEQ ID NO: 777 - DNA, 19 bp, homo sapiens
gcttttaatgcttaggaat

SEQ ID NO: 778 - DNA, 19 bp, homo sapiens
gcttaggaatatgtatatt

SEQ ID NO: 779 - DNA, 19 bp, homo sapiens
ccagtattccttgtatgaa

SEQ ID NO: 780 - DNA, 19 bp, homo sapiens
ccttgtatgaaaataatta

SEQ ID NO: 781 - DNA, 19 bp, homo sapiens
gctggtacctggtagcatt

SEQ ID NO: 782 - DNA, 19 bp, homo sapiens
ggtacctggtagcatttat

SEQ ID NO: 783 - DNA, 19 bp, homo sapiens
ggtagatgctctaccacat

SEQ ID NO: 784 - DNA, 19 bp, homo sapiens
ccacattccttcagctgta

SEQ ID NO: 785 - DNA, 19 bp, homo sapiens
gctgtattgagtgttccat

SEQ ID NO: 786 - DNA, 19 bp, homo sapiens
ccatttgcttatcaattta

SEQ ID NO: 787 - DNA, 19 bp, homo sapiens
cctcttttttttgtatgta

SEQ ID NO: 788 - DNA, 19 bp, homo sapiens
gcctgactccagcaattaa

SEQ ID NO: 789 - DNA, 19 bp, homo sapiens
cctgactccagcaattaat

SEQ ID NO: 790 - DNA, 19 bp, homo sapiens
gcggtgactcattgggctt

SEQ ID NO: 791 - DNA, 19 bp, homo sapiens
ggcttccagtacttctgtt

SEQ ID NO: 792 - DNA, 19 bp, homo sapiens
ccagtacttctgttgatta

SEQ ID NO: 793 - DNA, 19 bp, homo sapiens
cccaaaagaaagagcaaaa

SEQ ID NO: 794 - DNA, 19 bp, homo sapiens
ccaaaagaaagagcaaaat

SEQ ID NO: 795 - DNA, 19 bp, homo sapiens
gcaaaatggaaaatgcgat

SEQ ID NO: 796 - DNA, 19 bp, homo sapiens
gcgatccatgtcaccaaat

SEQ ID NO: 797 - DNA, 19 bp, homo sapiens
cgatccatgtcaccaaata

SEQ ID NO: 798 - DNA, 19 bp, homo sapiens
ccatgtcaccaaatataat

SEQ ID NO: 799 - DNA, 19 bp, homo sapiens
ccaaatataattgctacaa

SEQ ID NO: 800 - DNA, 19 bp, homo sapiens
gctacaagtaacatgaaat

SEQ ID NO: 801 - DNA, 19 bp, homo sapiens
gctcttgagcaattatata

SEQ ID NO: 802 - DNA, 19 bp, homo sapiens
ccttttaaaaaataagaaa

SEQ ID NO: 803 - DNA, 19 bp, homo sapiens
gctaggttttatcatacta

SEQ ID NO: 804 - DNA, 19 bp, homo sapiens
ggttttatcatactaaaat

SEQ ID NO: 805 - DNA, 19 bp, homo sapiens
ggttttttaaacttactaa

SEQ ID NO: 806 - DNA, 19 bp, homo sapiens
ccaaatacagttcttgaaa

SEQ ID NO: 807 - DNA, 19 bp, homo sapiens
gcttttagacttagtgcta

SEQ ID NO: 808 - DNA, 19 bp, homo sapiens
gccagggtacatcctatat

SEQ ID NO: 809 - DNA, 19 bp, homo sapiens
ccagggtacatcctatata

SEQ ID NO: 810 - DNA, 19 bp, homo sapiens
gggtacatcctatatacta

SEQ ID NO: 811 - DNA, 19 bp, homo sapiens
ccttaggtttcagtatttt

SEQ ID NO: 812 - DNA, 19 bp, homo sapiens
gctctgctgaaattaaaaa

SEQ ID NO: 813 - DNA, 19 bp, homo sapiens
gctgaaattaaaaaaaaat

SEQ ID NO: 814 - DNA, 19 bp, homo sapiens
gcagtggctcacacctgta

SEQ ID NO: 815 - DNA, 19 bp, homo sapiens
cctgtaatcccagcacttt

SEQ ID NO: 816 - DNA, 19 bp, homo sapiens
gccgaggtgggcagatcat

SEQ ID NO: 817 - DNA, 19 bp, homo sapiens
gcctgaccaacatggtgaa

SEQ ID NO: 818 - DNA, 19 bp, homo sapiens
cctgaccaacatggtgaaa

SEQ ID NO: 819 - DNA, 19 bp, homo sapiens
gccgggcatggtggcacat

SEQ ID NO: 820 - DNA, 19 bp, homo sapiens
ccgggcatggtggcacata

SEQ ID NO: 821 - DNA, 19 bp, homo sapiens
ggttgcagtgagccaagat

SEQ ID NO: 822 - DNA, 19 bp, homo sapiens
gcgagactccatctcaaaa

SEQ ID NO: 823 - DNA, 19 bp, homo sapiens
cgagactccatctcaaaaa

SEQ ID NO: 824 - DNA, 19 bp, homo sapiens
ccatagttctgaagtcttt

SEQ ID NO: 825 - DNA, 19 bp, homo sapiens
ggccatgaatgttctttaa

SEQ ID NO: 826 - DNA, 19 bp, homo sapiens
gccatgaatgttctttaat

SEQ ID NO: 827 - DNA, 19 bp, homo sapiens
ggtctaaaaagcggtttta

SEQ ID NO: 828 - DNA, 19 bp, homo sapiens
cctccatagcgtcaatgtt

SEQ ID NO: 829 - DNA, 19 bp, homo sapiens
gcgtcaatgttgacctatt

SEQ ID NO: 830 - DNA, 19 bp, homo sapiens
cgtcaatgttgacctattt

SEQ ID NO: 831 - DNA, 19 bp, homo sapiens
cctatttgatatattttta

SEQ ID NO: 832 - DNA, 19 bp, homo sapiens
gcagattatagatgcagaa

SEQ ID NO: 833 - DNA, 19 bp, homo sapiens
cctgtgatacaccaatgaa

SEQ ID NO: 834 - DNA, 19 bp, homo sapiens
ccaatgaaatattccaaaa

SEQ ID NO: 835 - DNA, 19 bp, homo sapiens
ggtatttgttaacttttaa

SEQ ID NO: 836 - DNA, 19 bp, homo sapiens
ggtttttattcttgtccaa

SEQ ID NO: 837 - DNA, 19 bp, homo sapiens
ccaacaatgaaataactta

SEQ ID NO: 838 - DNA, 19 bp, homo sapiens
gcatgggctaatggtggta

SEQ ID NO: 839 - DNA, 19 bp, homo sapiens
gggctaatggtggtatttt

SEQ ID NO: 840 - DNA, 19 bp, homo sapiens
ggctaatggtggtattttt

SEQ ID NO: 841 - DNA, 19 bp, homo sapiens
gctaatggtggtatttttt

SEQ ID NO: 842 - DNA, 19 bp, homo sapiens
cgccactgctgcgagggaa

SEQ ID NO: 843 - DNA, 19 bp, homo sapiens
gcctgtggctggctgagaa

SEQ ID NO: 844 - DNA, 19 bp, homo sapiens
ggctggctgagaagggcaa

SEQ ID NO: 845 - DNA, 19 bp, homo sapiens
ggcaaggtggatggctctt

SEQ ID NO: 846 - DNA, 19 bp, homo sapiens
gcagccagaggcagtgatt

SEQ ID NO: 847 - DNA, 19 bp, homo sapiens
ggcagtgattcacctcttt

SEQ ID NO: 848 - DNA, 19 bp, homo sapiens
gcagtgattcacctcttta

SEQ ID NO: 849 - DNA, 19 bp, homo sapiens
ggagtgaggtgggttttat

SEQ ID NO: 850 - DNA, 19 bp, homo sapiens
ggtgggttttattttcatt

SEQ ID NO: 851 - DNA, 19 bp, homo sapiens
gggttttattttcattttt

SEQ ID NO: 852 - DNA, 19 bp, homo sapiens
ggctgagttggggaatcat

SEQ ID NO: 853 - DNA, 19 bp, homo sapiens
gctgagttggggaatcatt

SEQ ID NO: 854 - DNA, 19 bp, homo sapiens
ggggaatcatttaaaattt

SEQ ID NO: 855 - DNA, 19 bp, homo sapiens
cccagctcccttctgccat

SEQ ID NO: 856 - DNA, 19 bp, homo sapiens
ccagctcccttctgccatt

SEQ ID NO: 857 - DNA, 19 bp, homo sapiens
ccttctgccatttacacaa

SEQ ID NO: 858 - DNA, 19 bp, homo sapiens
gccatttacacaaaatatt

SEQ ID NO: 859 - DNA, 19 bp, homo sapiens
ccatttacacaaaatattt

SEQ ID NO: 860 - DNA, 19 bp, homo sapiens
cgttgtggaactgagatat

SEQ ID NO: 861 - DNA, 19 bp, homo sapiens
ggaactgagatattgagta

SEQ ID NO: 862 - DNA, 19 bp, homo sapiens
cctcagatgggacaggcta

SEQ ID NO: 863 - DNA, 19 bp, homo sapiens
gggacaggctattggacaa

SEQ ID NO: 864 - DNA, 19 bp, homo sapiens
cctggttcatgaagtggaa

SEQ ID NO: 865 - DNA, 19 bp, homo sapiens
ggttcatgaagtggaattt

SEQ ID NO: 866 - DNA, 19 bp, homo sapiens
ggaatttcctttttgagta

SEQ ID NO: 867 - DNA, 19 bp, homo sapiens
cctttttgagtagtttctt

SEQ ID NO: 868 - DNA, 19 bp, homo sapiens
ccagacctactgtttgcaa

SEQ ID NO: 869 - DNA, 19 bp, homo sapiens
ccatctgtagagaatacaa

SEQ ID NO: 870 - DNA, 19 bp, homo sapiens
ggactgcccaggacttgtt

SEQ ID NO: 871 - DNA, 19 bp, homo sapiens
ggacttgttgcttgtcttt

SEQ ID NO: 872 - DNA, 19 bp, homo sapiens
gcttgtctttcatttcatt

SEQ ID NO: 873 - DNA, 19 bp, homo sapiens
gcattcaccttgaagtcaa

SEQ ID NO: 874 - DNA, 19 bp, homo sapiens
ccacatgccatgtcctctt

SEQ ID NO: 875 - DNA, 19 bp, homo sapiens
cctcttaacaagataagaa

SEQ ID NO: 876 - DNA, 19 bp, homo sapiens
gggaagctgacattctata

SEQ ID NO: 877 - DNA, 19 bp, homo sapiens
ggacattggaaagttgctt

SEQ ID NO: 878 - DNA, 19 bp, homo sapiens
ggcagcgtagactaaagtt

SEQ ID NO: 879 - DNA, 19 bp, homo sapiens
gcagcgtagactaaagtta

SEQ ID NO: 880 - DNA, 19 bp, homo sapiens
gcgtagactaaagttactt

SEQ ID NO: 881 - DNA, 19 bp, homo sapiens
cgtagactaaagttacttt

SEQ ID NO: 882 - DNA, 19 bp, homo sapiens
gcctcccccagttgtccaa

SEQ ID NO: 883 - DNA, 19 bp, homo sapiens
ggtgcagacatgaattgat

SEQ ID NO: 884 - DNA, 19 bp, homo sapiens
gcagacatgaattgatgaa

SEQ ID NO: 885 - DNA, 19 bp, homo sapiens
ggcctagggccacctagaa

SEQ ID NO: 886 - DNA, 19 bp, homo sapiens
gcctagggccacctagaat

SEQ ID NO: 887 - DNA, 19 bp, homo sapiens
cctagggccacctagaatt

SEQ ID NO: 888 - DNA, 19 bp, homo sapiens
ggttatttgtaaaacaaaa

SEQ ID NO: 889 - DNA, 19 bp, homo sapiens
ggactagggaatactataa

SEQ ID NO: 890 - DNA, 19 bp, homo sapiens
gggtggggtaccaggagaa

SEQ ID NO: 891 - DNA, 19 bp, homo sapiens
ggtggggtaccaggagaaa

SEQ ID NO: 892 - DNA, 19 bp, homo sapiens
ggtaccaggagaaagggaa

SEQ ID NO: 893 - DNA, 19 bp, homo sapiens
ggagaaagggaaccccctt

SEQ ID NO: 894 - DNA, 19 bp, homo sapiens
ccccctttctcctggtaaa

SEQ ID NO: 895 - DNA, 19 bp, homo sapiens
gggtaaggggggggataat

SEQ ID NO: 896 - DNA, 19 bp, homo sapiens
ggtacgaaatagtcacttt

SEQ ID NO: 897 - DNA, 19 bp, homo sapiens
gcctcattgaattcaggtt

SEQ ID NO: 898 - DNA, 19 bp, homo sapiens
cctcattgaattcaggttt

SEQ ID NO: 899 - DNA, 19 bp, homo sapiens
ggttttttaagtacttgaa

SEQ ID NO: 900 - DNA, 19 bp, homo sapiens
ccttattttagtttctttt

SEQ ID NO: 901 - DNA, 19 bp, homo sapiens
cctctttagcgtggattaa

SEQ ID NO: 902 - DNA, 19 bp, homo sapiens
gcatgagataccagcattt

SEQ ID NO: 903 - DNA, 19 bp, homo sapiens
gcatttttacaaatacttt

SEQ ID NO: 904 - DNA, 19 bp, homo sapiens
ccccaagcacatcttcttt

SEQ ID NO: 905 - DNA, 19 bp, homo sapiens
gcacatcttctttcctttt

SEQ ID NO: 906 - DNA, 19 bp, homo sapiens
gggaaaaaaacaaaacaaa

SEQ ID NO: 907 - DNA, 19 bp, homo sapiens
ggaaaaaaacaaaacaaaa

SEQ ID NO: 908 - DNA, 19 bp, homo sapiens
ggtaatccgtgtctttcaa

SEQ ID NO: 909 - DNA, 19 bp, homo sapiens
cgtgtctttcaaaaaacat

SEQ ID NO: 910 - DNA, 19 bp, homo sapiens
gggtgcttgtggcacgtat

SEQ ID NO: 911 - DNA, 19 bp, homo sapiens
gcttgtggcacgtatgtat

SEQ ID NO: 912 - DNA, 19 bp, homo sapiens
gcgggagggggatgagaat

SEQ ID NO: 913 - DNA, 19 bp, homo sapiens
cgggagggggatgagaatt

SEQ ID NO: 914 - DNA, 19 bp, homo sapiens
ccttcagtaggctgtaaaa

SEQ ID NO: 915 - DNA, 19 bp, homo sapiens
ggcattactgcatctgcta

SEQ ID NO: 916 - DNA, 19 bp, homo sapiens
cctgcctgccaacacactt

SEQ ID NO: 917 - DNA, 19 bp, homo sapiens
gcctgccaacacacttgat

SEQ ID NO: 918 - DNA, 19 bp, homo sapiens
gcaaacagccctcaagtat

SEQ ID NO: 919 - DNA, 19 bp, homo sapiens
cctcaagtatctgtcagat

SEQ ID NO: 920 - DNA, 19 bp, homo sapiens
cctatataaggtattgaat

SEQ ID NO: 921 - DNA, 19 bp, homo sapiens
ggataaaacttacttggtt

SEQ ID NO: 922 - DNA, 19 bp, homo sapiens
gccctatgttaagataatt

SEQ ID NO: 923 - DNA, 19 bp, homo sapiens
ccctatgttaagataatta

SEQ ID NO: 924 - DNA, 19 bp, homo sapiens
cctatgttaagataattat

SEQ ID NO: 925 - DNA, 19 bp, homo sapiens
ggctaatatcattttttat

SEQ ID NO: 926 - DNA, 19 bp, homo sapiens
gctaatatcattttttata

SEQ ID NO: 927 - DNA, 19 bp, homo sapiens
ggtatgtgtatatttggaa

SEQ ID NO: 928 - DNA, 19 bp, homo sapiens
ggttatttctgtactttat

SEQ ID NO: 929 - DNA, 19 bp, homo sapiens
cctaaatcacttgagactt

SEQ ID NO: 930 - DNA, 19 bp, homo sapiens
gctcttgacactaagggat

SEQ ID NO: 931 - DNA, 19 bp, homo sapiens
ccctgggggtgttccacta

SEQ ID NO: 932 - DNA, 19 bp, homo sapiens
gggtgttccactagggcat

SEQ ID NO: 933 - DNA, 19 bp, homo sapiens
ggtgttccactagggcatt

SEQ ID NO: 934 - DNA, 19 bp, homo sapiens
ccactagggcattactgta

SEQ ID NO: 935 - DNA, 19 bp, homo sapiens
gccacatagacttcaagat

SEQ ID NO: 936 - DNA, 19 bp, homo sapiens
ccacatagacttcaagata

SEQ ID NO: 937 - DNA, 19 bp, homo sapiens
ggcctatgttttattgcat

SEQ ID NO: 938 - DNA, 19 bp, homo sapiens
gcctatgttttattgcata

SEQ ID NO: 939 - DNA, 19 bp, homo sapiens
gcatagtgtgaaacgtgta

SEQ ID NO: 940 - DNA, 19 bp, homo sapiens
gcttggttaacctgtatat

SEQ ID NO: 941 - DNA, 19 bp, homo sapiens
ggttaacctgtatatagat

SEQ ID NO: 942 - DNA, 19 bp, homo sapiens
cctgtatatagatagctta

SEQ ID NO: 943 - DNA, 19 bp, homo sapiens
gcttattgttgactagtta

SEQ ID NO: 944 - DNA, 19 bp, homo sapiens
gggttgcctgtaatattta

SEQ ID NO: 945 - DNA, 19 bp, homo sapiens
ggttgcctgtaatatttaa

SEQ ID NO: 946 - DNA, 19 bp, homo sapiens
gcctgtaatatttaagctt

SEQ ID NO: 947 - DNA, 19 bp, homo sapiens
gctggtaggaacatataat

SEQ ID NO: 948 - DNA, 19 bp, homo sapiens
ggtaggaacatataatttt

SEQ ID NO: 949 - DNA, 19 bp, homo sapiens
ggaacatataatttttgta

SEQ ID NO: 950 - DNA, 19 bp, homo sapiens
gccttttttattttacaaa

SEQ ID NO: 951 - DNA, 19 bp, homo sapiens
ccttttttattttacaaat

SEQ ID NO: 952 - DNA, 19 bp, homo sapiens
ggaattccaatgtgttttt

SEQ ID NO: 953 - DNA, 19 bp, homo sapiens
gcttccgtgaggattaatt

SEQ ID NO: 954 - DNA, 19 bp, homo sapiens
cgtgaggattaatttggaa

SEQ ID NO: 955 - DNA, 19 bp, homo sapiens
ggattaatttggaaaggtt

SEQ ID NO: 956 - DNA, 19 bp, homo sapiens
gcttgagattgacttcaat